SEQUENCE LISTING (25 sequences)

SEQ ID NO: 1 - protein, 133 aa, synthetic construct
APTSSSTKKTQLQLEHLRLDLEMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIIRTLT

SEQ ID NO: 2 - protein, 133 aa, synthetic construct
APTSSSTKKTQLQLEHLRLDLEMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCTSIIRTLT

SEQ ID NO: 3 - protein, 133 aa, synthetic construct
APTSSSTKKTQLQLEHLRLDLEMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCTSIIRTLT

SEQ ID NO: 4 - protein, 133 aa, synthetic construct
APTSSSTKKTQLQLEHLRLDLEMILNGINNYKNPKLTRMLTAKFAMPKKATELKHLQCLEEELKPLEEVLNLAQSKNFHFDPRDVVSNINVFVLELKGSETTFMCEYADETATIVEFLNRWITFCTSIIRTLT

SEQ ID NO: 5 - protein, 133 aa, synthetic construct
APTSSSTKKTQLQLEHLRLDLEMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIISTLT

SEQ ID NO: 6 - protein, 133 aa, synthetic construct
APTSSSTKKTQLQLEHLRLDLEMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCTSIISTLT

SEQ ID NO: 7 - protein, 133 aa, synthetic construct
APTSSSTKKTQLQLEHLRLDLEMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCTSIISTLT

SEQ ID NO: 8 - protein, 133 aa, synthetic construct [sequence omitted]

SEQ ID NO: 9 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 10 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRELIEELINITQNQKAPLCNGSMVWSINLTAGMYCAALESLINVSGCSAIEKTQRMLSGFCPHKVSAGQFSSLHVRSSKIEVAQFVKDLLFHLRTLFREGQFN

SEQ ID NO: 11 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 149 aa, synthetic construct
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 13 - protein, 169 aa, synthetic construct
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 14 - protein, 10 aa, synthetic construct
GGGGSGGGGS

SEQ ID NO: 15 - protein, 15 aa, synthetic construct
GGGGSGGGGSGGGGS

SEQ ID NO: 16 - protein, 20 aa, synthetic construct
GGGGSGGGGSGGGGSGGGGS

SEQ ID NO: 17 - protein, 20 aa, synthetic construct
GGGGSGGGGSGGGGSGGGGS

SEQ ID NO: 18 - protein, 733 aa, synthetic construct
APTSSSTKKTQLQLEHLRLDLEMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIIRTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 518 aa, synthetic construct
APTSSSTKKTQLQLEHLRLDLEMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIIRTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSS

SEQ ID NO: 20 - protein, 518 aa, synthetic construct
APTSSSTKKTQLQLEHLRLDLEMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCTSIIRTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSS

SEQ ID NO: 21 - protein, 518 aa, synthetic construct
APTSSSTKKTQLQLEHLRLDLEMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIIRTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLRVIMQSKWFKCGA

SEQ ID NO: 22 - protein, 518 aa, synthetic construct
APTSSSTKKTQLQLEHLRLDLEMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCTSIIRTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLRVIMQSKWFKCGA

SEQ ID NO: 23 - protein, 128 aa, synthetic construct
KCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSS

SEQ ID NO: 24 - protein, 134 aa, synthetic construct
MDTTEKETFCRAATVLRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAAS

SEQ ID NO: 25 - protein, 129 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLRVIMQSKWFKCGA

(19) United States
(12) Patent Application Publication
(10) Pub. No.: US 2026/0226125 A1
(43) Pub. Date: Aug. 6, 2026

(54) IL-2 SUPERANTAGONISTS CONSTRUCTS, METHODS AND USES THEREOF

(71) Applicant: Medicenna Therapeutics Inc., Toronto (CA)

(72) Inventor: Fahar Merchant, Vancouver (CA)

(21) Appl. No.: 19/110,294

(22) PCT Filed: Sep. 14, 2023
(86) PCT No.: PCT/IB2023/000548

§ 371 Date: Mar. 10, 2025

Related U.S. Application Data
(60) Provisional application No. 63/375,675, filed on Sep. 14, 2022.

(51) Int. Cl. C07K 14/55; A61K 38/00

(52) U.S. Cl. CPC C07K 14/55; A61K 38/00; C07K 2319/30; C07K 2319/31

(57) ABSTRACT

Human interleukin-2 (IL-2) muteins or variants thereof are provided. Also provided are pharmaceutical compositions that include such IL-2 muteins.

### CROSS-REFERENCE TO RELATED APPLICATIONS

This application is a U.S. national phase under 37 CFR § 3.71 of International Application No. PCT/IB2023/000548, filed Sep. 14, 2023, which claims priority to U.S. Provisional Application No. 63/375,675, filed Sep. 14, 2022, which is herein incorporated by reference in its entirety.

### SEQUENCE LISTING

The instant application contains a Sequence Listing which has been submitted electronically in XML file format and is hereby incorporated by reference in its entirety. Said XML copy, created on Apr. 14, 2026, is named 2026-04-14_117802-5016-WO_ST26_SeqList.xml and is 28,056 bytes in size.

### BACKGROUND

Interleukin 2 (IL-2) is a pluripotent cytokine produced primarily by activated CD4+ T cells, which plays a crucial role in producing a normal immune response. IL-2 promotes proliferation and expansion of activated T lymphocytes, potentiates B cell growth, and activates monocytes and natural killer cells. It was by virtue of these activities that IL-2 was tested and is used as an approved treatment of cancer (aldesleukin, Proleukin®). In eukaryotic cells, human IL-2 is synthesized as a precursor polypeptide of 153 amino acids, from which 20 amino acids are removed to generate mature secreted IL-2 (Taniguchi 1983). Recombinant human IL-2 has been produced in E. coli (Rosenberg 1984), in insect cells (Smith 1985) and in mammalian COS cells (Taniguchi 1983).

Interleukin-2 (IL-2) is a four α-helical bundle type I cytokine first identified as a T cell growth factor (Morgan et al., Science 193:1007 (1976)) but subsequently shown to have broad actions. IL-2 promotes T helper differentiation (Zhu et al., Annual review of immunology 28:445 (2010); Liao et al., Nat Immunol 9:1288 (2008); and Liao et al., Nat Immunol 12:551 (2011)) and the development of regulatory T (Treg) cells (Cheng et al., Immunol Rev 241:63 (2011)), induces natural killer and lymphokine activated killer activity (Liao et al., Immunity 38:13 (2013)), and mediates activation-induced cell death (AICD) (Lenardo et al., Nature 353:858 (1991)).

IL-2 works by interacting with three different receptors: the interleukin 2 receptor alpha (IL-2Rα; CD25), the interleukin 2 receptor beta (IL-2RB; CD122), and the interleukin 2 receptor gamma (IL-2Rγ; CD132; common gamma chain). The first receptor to be identified was the IL-2Rα, which is a 55 kD polypeptide (p55) that appears upon T cell activation and was originally called Tac (for T activation) antigen. The IL-2Rα binds IL-2 with a K_{d} of approximately 10⁻⁸ M and is also known as the “low affinity” IL-2 receptor. Binding of IL-2 to cells expressing only the IL-2Rα does not lead to any detectable biologic response. In most circumstances, IL-2 works through three different receptors: the IL-2Rα, the IL-2Rβ, and the IL-2Rγ. Most cells, such as resting T cells, are not responsive to IL-2 since they only express the IL-2Rβ, and the IL-2Rγ, which have low affinity for IL-2. Upon stimulation, resting T cells express the relatively high affinity IL-2 receptor IL-2Rα. Binding of IL-2 to the IL-2Rα causes this receptor to sequentially engage the IL-2Rβ, and the IL-2Rγ, bringing about T cell activation. IL-2 “superkines” with augmented action due to enhanced binding affinity for IL-2Rβ were previously developed (Levin et al., Nature 484:529 (2012)).

Despite the wealth of knowledge around IL-2, including IL-2 superantagonists, there remains a need in the art for better therapies for the treatment of cancer, including new therapies comprising IL-2 muteins as provided herein.

### BRIEF SUMMARY

IL-2 exerts a wide spectrum of effects on the immune system, and it plays crucial roles in regulating both immune activation and homeostasis and finds use in the treatment of cancer.

The present invention provides for an IL-2 mutein comprising amino acid substitutions L18R, Q22E, and Q126T numbered in accordance with wild-type human IL-2 (hIL-2) (SEQ ID NO: 8), and further comprising a group of amino acid substitutions selected from the group consisting of F42A, Y45A, E62A, L80F, R81D, L85V, I86V, I92F, Q126T, and S130R.

In some embodiments, the IL-2 mutein comprises the amino acid substitutions L18R, Q22E, L80F, R81D, L85V, I86V, I92F, Q126T, and S130R, optionally wherein the IL-2 mutein comprises an amino acid sequence of SEQ ID NO: 1.

In some embodiments, the IL-2 mutein comprises the amino acid substitutions L18R, Q22E, L80F, R81D, L85V, I86V, I92F, Q126T, S130R, F42A, and E62A, optionally wherein the IL-2 mutein comprises an amino acid sequence of SEQ ID NO: 2.

In some embodiments, the IL-2 mutein comprises the amino acid substitutions L18R, Q22E, L80F, R81D, L85V, I86V, I92F, Q126T, S130R, and F42A, optionally wherein the IL-2 mutein comprises an amino acid sequence of SEQ ID NO: 3.

In some embodiments, the IL-2 mutein comprises the amino acid substitutions L18R, Q22E, L80F, R81D, L85V, I86V, I92F, Q126T, S130R, F42A, and Y45A, optionally wherein the IL-2 mutein comprises an amino acid sequence of SEQ ID NO: 4.

In some embodiments, the IL-2 mutein comprises the amino acid substitutions L18R, Q22E, L80F, R81D, L85V, I86V, I92F, and Q126T, optionally wherein the IL-2 mutein comprises an amino acid sequence of SEQ ID NO: 5.

In some embodiments, the IL-2 mutein comprises the amino acid substitutions L18R, Q22E, L80F, R81D, L85V, I86V, I92F, Q126T, F42A, and E62A, optionally wherein the IL-2 mutein comprises an amino acid sequence of SEQ ID NO: 6.

In some embodiments, the IL-2 mutein comprises the amino acid substitutions L18R, Q22E, L80F, R81D, L85V, I86V, I92F, Q126T, and F42A, optionally wherein the IL-2 mutein comprises an amino acid sequence of SEQ ID NO: 7.

In some embodiments, the IL-2 mutein is fused to an albumin molecule, an Fc molecule, and/or another mutein, optionally wherein the other mutein is an IL-13 mutein or an IL-4 mutein.

In some embodiments, the IL-2 mutein is fused to an albumin molecule.

In some embodiments, the fusion protein comprises an amino acid sequence of SEQ ID NO: 18.

In some embodiments, the IL-2 mutein is fused to an Fc molecule.

In some embodiments, the IL-2 mutein is fused to an Fc molecule and an IL-13 mutein, optionally wherein the IL-13 mutein comprises the amino acid substitutions L10V, V181, D87S, T88S, L101F, K104R, and K105T (A11) numbered in accordance with wild-type human IL-13 (hIL-13).

In some embodiments, the IL-2 mutein is fused to an Fc molecule and an IL-4 mutein, optionally wherein the IL-4 mutein comprises the amino acid substitutions R121K, Y124F, and S125R (KFR) or K117R, T118V, R121Q, E122S, Y124W, S125F, S128G, and S129A (RGA) numbered in accordance with wild-type human IL-4 (hIL-4).

In some embodiments, the fusion protein comprises an amino acid sequence of one of SEQ ID NOs: 19-22.

In some embodiments, the IL-2 mutein has increased binding to CD122, compared to wild-type IL-2.

In some embodiments, the IL-2 mutein has decreased binding to CD25, compared to wild-type IL-2.

In some embodiments, the IL-2 mutein has inhibitory activity, as determined using a HEKBlue IL-2 and/or a CTLL2 assay.

In some embodiments, the IL-2 mutein inhibits IL-2 induced pSTAT5 signaling in human PBMCs.

In some embodiments, the IL-2 mutein is not toxic in mice, as determined using a maximum tolerated dose (MTD) assay.

In some embodiments, the IL-2 mutein reduces disease scores, as determined using Experimental Autoimmune Encephalomyelitis (EAE) analysis.

The present invention also provides nucleic acids encoding the IL-2 mutein as described herein.

The present invention also provides vectors comprising the nucleic acids as described herein.

The present invention also provides host cells comprising the nucleic acids or the vectors as described herein.

### BRIEF DESCRIPTION OF THE DRAWINGS

FIG. 1: Schematic of MDNA209, an IL-2/IL-15 antagonist with a unique mechanism of action

FIGS. 2A-2D: SPR sensorgrams of IL-2 antagonist constructs binding to IL-2R βγc Complex (FIG. 2A) and to human CD25 and CD122 (FIGS. 2B-D).

FIGS. 3A-2C. Representative dose response curves of various MDNA209 antagonist molecules in HEK-Blue™ IL-2 reporter assay. Compounds were tested in the agonist (A, C) and antagonist formats (B). In FIG. 2A, the EC₈₀ value for IL-2 activation and background levels are shown by the upper and lower dotted lines, respectively.

FIG. 4: Representative graph of dose response of IL-2-Fc alone or in the presence of 30 nM MDNA209-Fc in the CTLL2 proliferation assay. A four-parameter logistic curve fit is presented as a solid line. Error bars represent the standard error of the mean from replicate wells.

FIGS. 5A-5B: Representative dose response curves of percent pSTAT5 in response to antagonists in the presence of 67 μM rhIL-2 (FIG. 5A) or 670 μM rhIL-2 (FIG. 5B).

FIG. 6. Effect of MDNA209FEAA-Fc-A11 (30 mg/kg) and A11-Fc (21 and 11 mg/kg) on body weight in C57BL/6J mice.

FIG. 7. EAE scores (mean±SEM) for mice treated with PBS, MDNA209FA-Fc or MDNA209-Fc at peak disease. Mice were treated on day 8, 12, and 15 after immunization. * denotes significant difference (t-test, p=0.016, n=7, mean±SEM).

FIG. 8. EAE scores and body weight for mice treated with PBS, MDNA209FEAA-Fc-A11 (20 mg/kg) or a combination of MDNA209-Fc & A11-Fc (15.15 mg/kg & 14.1 mg/kg). Mice were treated on day 8, 12 and 15 after immunization. Data is shown as mean±SEM and * denotes significant differences p<0.05, t-test. MDNA209-Fc & A11-Fc combinations n=7 for Day 6-10, n=6 for Day 12, n=1 for day 15 onward; for MDNA209FEAA-Fc-A11 n=7 for Day 6-14 and n=4 from Day 15 onward. Scores from mice that died were carried forward.

FIG. 9. EAE scores for mice treated with PBS or MDNA209FEAA-Fc-A11 with a therapeutic dosing schedule. Mice were treated on day 12, 16 and 19 post immunization with 20 mg/kg and 5 mg/kg as indicated (dotted lines). Data is shown as mean±SEM, n=7 for PBS treated and n=5 for MDNA209FEAA-Fc-A11 treatment.

FIG. 10. IFNγ levels quantified by ELISA of PBMC treated with increasing concentration of MDNA209-Fc in the presence of indicated rhIL-2 concentration for 48 hours.

FIG. 11. MDNA209-Fc induced a dose-dependent inhibition of IFNγ production (shown as percent of maximum) in human PBMCs stimulated with 0.3 μg/ml of rhIL-2 from four different donors.

FIGS. 12A-12D. Body weight (FIGS. 12A-12B) and total lymphocyte count (FIGS. 12C-12D) of animals receiving PBS or MDNA209-Fc.

FIG. 13. MDNA209-Fc detection ELISA was performed on blood plasma collected at 5 min, 24 h or 72 h post-injections of MDNA-Fc (10 mg/kg and 20 mg/kg) (N=3/group).

FIG. 14. MDNA209-Fc inhibits the proliferation of PBMCs in MLR assay. Representative non-linear fit curves of stimulation index (SI) for MDNA209-Fc, MDNA209-albumin and MDNA209FEAA-Fc plotted against respective test dose range.

FIGS. 15A-15B. Sensorgrams of IL-2 antagonists binding to human IL2Rα (CD25) and IL2Rβ (CD122).

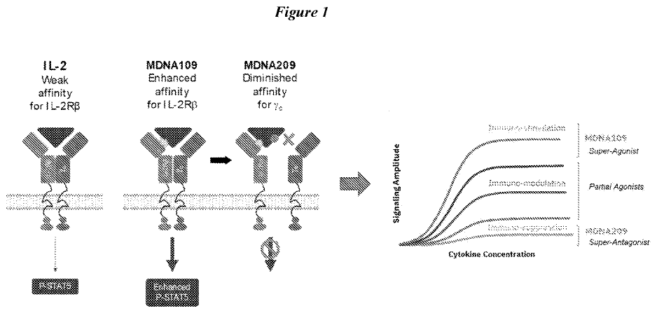

### DETAILED DESCRIPTION

In order for the present disclosure to be more readily understood, certain terms and phrases are defined below as well as throughout the specification.

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russell, Molecular Cloning: A Laboratory Manual 3rd ed., Cold Spring harbor Laboratory Press (Cold Spring Harbor, NY 2001), provide one skilled in the art with a general guide to many terms used in the present disclosure. As appropriate, procedures involving the use of commercially available kits and reagents are generally carried out in accordance with manufacturer defined protocols and/or parameters unless otherwise noted.

As used herein, “IL-2” means wild-type IL-2, whether native or recombinant. Mature human IL-2 occurs as a 133 amino acid sequence (less the signal peptide, consisting of an additional 20 N-terminal amino acids), as described in Fujita, et. al., PNAS USA, 80, 7437-7441 (1983). The amino acid sequence of human IL-2 (SEQ ID NO: 11; full length) is found in Genbank under accession locator NP_000577.2. The amino acid sequence of mature human IL-2 is depicted in SEQ ID NO: 8 (human wild-type mature; position numbering of the substitutions is based on this sequence). The murine (Mus musculus) IL-2 amino acid sequence is found in Genbank under accession locator (SEQ ID NO: 13). The amino acid sequence of mature murine IL-2 is depicted in SEQ ID NO: 12.

SEQ ID NO: 11
MYRMQLLSCIALSLALVTNSAPTSSSTKKTQLQLEHLLLDLQMILNGINNYKNPKLTRMLT
FKFYMPKKATELKHLQCLEEELKPLEEVLNLAQSKNFHLRPRDLISNINVIVLELKGSETTF
MCEYADETATIVEFLNRWITFCQSIISTLT
SEQ ID NO: 8
APTSSSTKKTQLQLEHLLLDLQMILNGINNYKNPKLTRMLTFKFYMPKKATELKHLQCLEE
ELKPLEEVLNLAQSKNFHLRPRDLISNINVIVLELKGSETTFMCEYADETATIVEFLNRWITF
CQSIISTLT
SEQ ID NO: 13
MYSMQLASCVTLTLVLLVNSAPTSSSTSSSTAEAQQQQQQQQQQQQHLEQLLMDLQELLS
RMENYRNLKLPRMLTFKFYLPKQATELKDLQCLEDELGPLRHVLDLTQSKSFQLEDAENFI
SNIRVTVVKLKGSDNTFECQFDDESATVVDFLRRWIAFCQSIISTSPQ
SEQ ID NO: 12
APTSSSTSSSTAEAQQQQQQQQQQQQHLEQLLMDLQELLSRMENYRNLKLPRMLTFKFYL
PKQATELKDLQCLEDELGPLRHVLDLTQSKSFQLEDAENFISNIRVTVVKLKGSDNTFECQF
DDESATVVDFLRRWIAFCQSIISTSPQ

As used herein, “IL-2 mutein” means an IL-2 polypeptide wherein specific substitutions to the interleukin-2 protein have been made. The IL-2 muteins are characterized by amino acid insertions, deletions, substitutions and modifications at one or more sites in or at the other residues of the native IL-2 polypeptide chain. In accordance with this disclosure, any such insertions, deletions, substitutions and modifications result in an IL-2 mutein that retains the IL-2Rβ binding activity. Exemplary muteins can include substitutions of 1, 2, 3, 4, 5, 6, 7, 8, 9, 10 or more amino acids.

Muteins also include conservative modifications and substitutions at other positions of IL-2 (i.e., those that have a minimal effect on the secondary or tertiary structure of the mutein). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gln, asn, ser, thr; Group II: cys, ser, tyr, thr; Group III: val, ile, leu, met, ala, phe; Group IV: lys, arg, his; Group V: phe, tyr, trp, his; and Group VI: asp, glu.

“Numbered in accordance with IL-2” means identifying a chosen amino acid with reference to the position at which that amino acid normally occurs in the mature sequence of wild type IL-2, for example R81 refers to the eighty-first amino acid, arginine, that occurs in SEQ ID NO: 8. L80 refers to the eightieth amino acid, leucine, that occurs in SEQ ID NO: 8. L85 refers to the eighty-fifth amino acid, leucine, that occurs in SEQ ID NO: 8. 186 refers to the eighty-sixth amino acid, isoleucine, that occurs in SEQ ID NO: 8. 192 refers to the ninety-second amino acid, isoleucine, that occurs in SEQ ID NO: 8. F42 refers to the forty-second amino acid, phenylalanine, that occurs in SEQ ID NO: 8. K43 refers to the forty-third amino acid, lysine, that occurs in SEQ ID NO: 8. L18 refers to the eighteenth amino acid, leucine, that occurs in SEQ ID NO: 8. Q22 refers to the twenty-second amino acid, glutamine, that occurs in SEQ ID NO: 8. Q126 refers to the one hundred twenty-sixth amino acid, glutamine, that occurs in SEQ ID NO: 8. S130 refers to the one hundred thirtieth amino acid, serine, that occurs in SEQ ID NO: 8. E62 refers to the sixty-second amino acid, glutamic acid, that occurs in SEQ ID NO: 8. Y45 refers to the forty-fifth amino acid, tyrosine, that occurs in SEQ ID NO: 8.

As used herein, the abbreviations for the genetically encoded L-enantiomeric amino acids used in the disclosure methods are conventional and are as follows in Table 1.

TABLE 1
Amino acid abbreviations

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R).

The term “cell types having the IL-2Rαβγ receptor” means the cells known to have this receptor type, i.e., T cells, activated T cells, B cells, activated monocytes, and activated NK cells. The term “cell types having the IL-2Rβγ receptor” means the cells known to have that receptor type, i.e., B cells, resting monocytes, and resting NK cells.

The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The terms “polypeptide,” “protein” or “peptide” refer to any chain of amino acid residues, regardless of its length or post-translational modification (e.g., glycosylation or phosphorylation).

In the event the mutant IL-2 polypeptides of the disclosure are “substantially pure,” they can be at least about 60% by weight (dry weight) the polypeptide of interest, for example, a polypeptide containing the mutant IL-2 amino acid sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

An “agonist” is a compound that interacts with a target to cause or promote an increase in the activation of the target.

A “partial agonist” is a compound that interacts with the same target as an agonist but does not produce as great a magnitude of a biochemical and/or physiological effect as the agonist, even by increasing the dosage of the partial agonist.

A “superagonist” (also referred to as a “superkine”) is a type of agonist that is capable of producing a maximal response greater than the endogenous agonist for the target receptor, and thus has an efficacy of more than 100%.

“Operably linked” is intended to mean that the nucleotide sequence of interest (i.e., a sequence encoding an IL-2 mutein) is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression constructs of the invention can be introduced into host cells to thereby produce the human IL-2 muteins disclosed herein or to produce biologically active variants thereof.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

As used herein, the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, reproductive systems, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus. Cancers generally can include prostate cancer, ovarian cancer, breast cancer, endometrial cancer, multiple myeloma, melanoma, lymphomas, lung cancers including small cell lung cancer, kidney cancer, colorectal cancer, pancreatic cancer, gastric cancer, and brain cancer.

The term “carcinoma” is art-recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

As used herein, the term “hematopoietic neoplastic disorders” refers to diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Stemberg disease.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease. A therapeutically effective amount can be an amount that reduces tumor number, tumor size, and/or increases survival.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an IL-2 mutein to a subject. In some embodiments, the IL-2 mutein administered further comprises a substitution at position F42A. In some embodiments, the IL-2 administered mutein further comprises a substitution at position K43N.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, the therapeutic effect is to reduce tumor number. In some embodiments, the therapeutic effect is to reduce tumor size. In some embodiments, the therapeutic effect is to increase survival.

In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. IL-2 muteins and pharmaceutical compositions thereof can be packaged in a single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue or vasculature of a subject (for example, liver tissue or veins). Other effective dosages can be readily established by one of ordinary skill in the art through routine trials establishing dose response curves.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome. In some embodiments, the effective amount is an amount sufficient to reduce tumor number. In some embodiments, the effective amount is an amount sufficient to reduce tumor size. In some embodiments, the effective amount is an amount sufficient to increase survival.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

### I. Detailed Description

Described herein IL-2 muteins comprising the amino acid substitutions L18R, Q22E, and Q126T numbered in accordance with wild-type human IL-2 (hIL-2) (SEQ ID NO: 8), and further comprising a group of amino acid substitutions selected from the group consisting of F42A, Y45A, E62A, L80F, R81D, L85V, I86V, I92F, Q126T, and S130R. Such IL-2 muteins find use, for example, in treatment of cancer. Also provided are nucleic acids encoding such IL-2 muteins, methods of making such IL-2 muteins, pharmaceutical compositions that include such IL-2 muteins and methods of treatment using such IL-2 muteins.

### A. IL-2 Muteins

The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations can be at amino acid residues that contact the IL-2Rβ and/or the IL-2Rγ.

More specifically, a mutation (whether conservative or non-conservative, by way of addition(s) or deletion(s)) can be made at one or more of positions. For example, the mutations can be L18R, Q22E, and Q126T, and can further comprise a group of mutations selected from the group consisting of F42A, Y45A, E62A, L80F, R81D, L85V, I86V, I92F, Q126T, and S130R.

For example, the mutations can be: L18R, Q22E, L80F, R81D, L85V, I86V, I92F, Q126T, S130R, F42A, and E62A.

For example, the mutations can be: L18R, Q22E, L80F, R81D, L85V, I86V, I92F, Q126T, S130R, and F42A.

For example, the mutations can be: L18R, Q22E, L80F, R81D, L85V, I86V, I92F, Q126T, S130R, F42A, and Y45A.

For example, the mutations can be: L18R, Q22E, L80F, R81D, L85V, I86V, I92F, and Q126T.

For example, the mutations can be: L18R, Q22E, L80F, R81D, L85V, I86V, I92F, Q126T, F42A, and E62A.

For example, the mutations can be: L18R, Q22E, L80F, R81D, L85V, I86V, I92F, Q126T, and F42A.

TABLE 2
List of MDNA209 Variants analyzed

| SEQ ID NO: | |
|---|---|
| (Information) | Amino acid sequence |
| SEQ ID NO: 1 | APTSSSTKKTQLQLEHLRLDLEMILNGINNYKNP |
| (MDNA209; Mutations: L18R, Q22E, | KLTRMLTFKFYMPKKATELKHLQCLEEELKPLEE |
| L80F, R81D, L85V, I86V, 192F, | VLNLAQSKNFHFDPRDVVSNINVFVLELKGSETT |
| Q126T, S130R) | FMCEYADETATIVEFLNRWITFCTSIIRTLT |
| SEQ ID NO: 2 | APTSSSTKKTQLQLEHLRLDLEMILNGINNYKNPK |
| (MDNA209FEAA; Mutations: L18R, | LTRMLTAKFYMPKKATELKHLQCLEEALKPLEEVL |
| Q22E, L80F, R81D, L85V, I86V, 192F, | NLAQSKNFHFDPRDVVSNINVFVLELKGSETTFMC |
| Q126T, S130R, F42A, E62A) | EYADETATIVEFLNRWITFCTSIIRTLT |
| SEQ ID NO: 3 | APTSSSTKKTQLQLEHLRLDLEMILNGINNYKNPK |
| (MDNA209FA; Mutations: L18R, | LTRMLTAKFYMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T, S130R, F42A) | EYADETATIVEFLNRWITFCTSIIRTLT |
| SEQ ID NO: 4 | APTSSSTKKTQLQLEHLRLDLEMILNGINNYKNPK |
| (MDNA209FEY; Mutations: L18R, | LTRMLTAKFAMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T, S130R, F42A, Y45A) | EYADETATIVEFLNRWITFCTSIIRTLT |
| SEQ ID NO: 5 | APTSSSTKKTQLQLEHLRLDLEMILNGINNYKNPK |
| (MDNA209(3); Mutations: L18R, | LTRMLTFKFYMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T) | EYADETATIVEFLNRWITFCTSIISTLT |
| SEQ ID NO: 6 | APTSSSTKKTQLQLEHLRLDLEMILNGINNYKNPK |
| (MDNA209(3)FEAA; Mutations: | LTRMLTAKFYMPKKATELKHLQCLEEALKPLEEVL |
| L18R, Q22E, L80F, R81D, L85V, | NLAQSKNFHFDPRDVVSNINVFVLELKGSETTFMC |
| I86V, I92F, Q126T, F42A, E62A) | EYADETATIVEFLNRWITFCTSIISTLT |
| SEQ ID NO: 7 | APTSSSTKKTQLQLEHLRLDLEMILNGINNYKNPK |
| (MDNA209(3)FA; Mutations: L18R, | LTRMLTAKFYMPKKATELKHLQCLEEELKPLEEVL |
| Q22E, L80F, R81D, L85V, I86V, I92F, | NLAQSKNFHFDPRDVVSNINVFVLELKGSETTFMC |
| Q126T, F42A) | EYADETATIVEFLNRWITFCTSIISTLT |
| SEQ ID NO: 8 | APTSSSTKKTQLQLEHLLLDLQMILNGINNYKNPK |
| (human wild-type IL-2) | LTRMLTFKFYMPKKATELKHLQCLEEELKPLEEVL |
| | NLAQSKNFHLRPRDLISNINVIVLELKGSETTFMC |
| | EYADETATIVEFLNRWITFCQSIISTLT |

In some embodiments, the substitutions in the IL-2 mutein comprise L18R, Q22E, and Q126T, and further comprise a group of amino acid substitutions selected from the group consisting of F42A, Y45A, E62A, L80F, R81D, L85V, I86V, I92F, Q126T, and S130R, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, and S130R, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, S130R, F42A, and E62A, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, S130R, and F42A, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, S130R, F42A, and Y45A, numbered in accordance with wild-type human IL-2 of SEQ ID NO: 8. In some embodiments, the substitutions in the IL-2 mutein comprise L18R, Q22E, L80F, R81D, L85V, I86V, I92F, and Q126T, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, F42A, and E62A, numbered in accordance with wild-type human IL-2 of SEQ ID NO: 8. In some embodiments, the substitutions in the IL-2 mutein comprise L18R, Q22E, L80F, R81D, L85V, I86V, I92F, Q126T, and F42A, numbered in accordance with wild-type human IL-2 of SEQ ID NO: 8.

In some embodiments, the substitutions in the IL-2 mutein that lead to increased and/or enhanced IL-2Rβ binding include L18R, Q22E, L80F, R81D, L85V, I86V, I92F, and Q126T, numbered in accordance with wild-type human IL-2 of SEQ ID NO: 8. In some embodiments, an IL-2 mutein for use in the invention comprises L18R, Q22E, L80F, R81D, L85V, I86V, I92F, and Q126T, and exhibits increased IL-2Rβ binding. In some embodiments, an IL-2 mutein for use in the invention further comprises a substitution at position S130R. In some embodiments, an IL-2 mutein for use in the invention further comprises a substitution at position S130R. In some embodiments, an IL-2 mutein for use in the invention further comprises a substitution at position F42A. In some embodiments, an IL-2 mutein for use in the invention further comprises a substitution at position E62A. In some embodiments, an IL-2 mutein for use in the invention further comprises a substitution at position Y45A. In some embodiments, the substitutions in the IL-2 mutein comprise L18R, Q22E, and Q126T, and further comprise a group of amino acid substitutions selected from the group consisting of F42A, Y45A, E62A, L80F, R81D, L85V, I86V, I92F, Q126T, and S130R, numbered in accordance with wild-type human IL-2 of SEQ ID NO: 8. In some embodiments, the IL-2 mutein comprises substitutions L18R, Q22E, L80F, R81D, L85V, I86V, I92F, Q126T, and S130R, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, F42A, and E62A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, and F42A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, F42A, and Y45A, numbered in accordance with wild-type human IL-2 of SEQ ID NO: 8. In some embodiments, the IL-2 mutein comprises substitutions L18R, Q22E, L80F, R81D, L85V, I86V, I92F, and Q126T, numbered in accordance with wild-type human IL-2 of SEQ ID NO: 8. In some embodiments, the IL-2 mutein comprises substitutions L18R, Q22E, L80F, R81D, L85V, I86V, I92F, Q126T, F42A, and E62A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, and F42A, numbered in accordance with wild-type human IL-2 of SEQ ID NO: 8.

In some embodiments, the mutein comprises substitutions L18R, Q22E, and Q126T, and one or more substitutions selected from the group consisting of F42A, Y45A, E62A, L80F, R81D, L85V, I86V, I92F, Q126T, and S130R, all as compared to wild-type human IL-2 (SEQ ID NO: 8).

In some embodiments, the amino acid substitutions increasing IL-2Rβ binding affinity include: L18R, Q22E, L80F, R81D, L85V, I86V, I92F, Q126T, S130R, F42A, E62A and/or Y45A. In some embodiments, the amino acid substitutions that increase IL-2Rβ binding affinity include: L18R, Q22E, L80F, R81D, L85V, I86V, I92F, Q126T, S130R, F42A, E62A and/or Y45A.

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, and S130R. In some embodiments, the IL-2 mutein has the amino acid sequence:

(SEQ ID NO: 1)
APTSSSTKKTQLQLEHLRLDLEMILNGINNYKNPKLTRMLTFKFYMPKKATELKHLQCLEE
ELKPLEEVLNLAQSKNFHFDPRDVVSNINVFVLELKGSETTFMCEYADETATIVEFLNRWI
TFCTSIIRTLT.

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, S130R, F42A, and E62A. In some embodiments, the IL-2 mutein has the amino acid sequence:

(SEQ ID NO: 2)
APTSSSTKKTQLQLEHLRLDLEMILNGINNYKNPKLTRMLTAKFYMPKKATELKHLQCLE
EALKPLEEVLNLAQSKNFHFDPRDVVSNINVFVLELKGSETTFMCEYADETATIVEFLNRW
ITFCTSIIRTLT

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, S130R, and F42A. In some embodiments, the IL-2 mutein has the amino acid sequence:

(SEQ ID NO: 3)
APTSSSTKKTQLQLEHLRLDLEMILNGINNYKNPKLTRMLTAKFYMPKKATELKHLQCLE
EELKPLEEVLNLAQSKNFHFDPRDVVSNINVFVLELKGSETTFMCEYADETATIVEFLNRW
ITFCTSIIRTLT

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, S130R, F42A, and Y45A. In some embodiments, the IL-2 mutein has the amino acid sequence:

(SEQ ID NO: 4)
APTSSSTKKTQLQLEHLRLDLEMILNGINNYKNPKLTRMLTAKFAMPKKATELKHLQCLE
EELKPLEEVLNLAQSKNFHFDPRDVVSNINVFVLELKGSETTFMCEYADETATIVEFLNRW
ITFCTSIIRTLT

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, and Q126T. In some embodiments, the IL-2 mutein has the amino acid sequence:

(SEQ ID NO: 5)
APTSSSTKKTQLQLEHLRLDLEMILNGINNYKNPKLTRMLTFKFYMPKKATELKHLQCLEE
ELKPLEEVLNLAQSKNFHFDPRDVVSNINVFVLELKGSETTFMCEYADETATIVEFLNRWI
TFCTSIISTLT

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, F42A, and E62A. In some embodiments, the IL-2 mutein has the amino acid sequence:

(SEQ ID NO: 6)
APTSSSTKKTQLQLEHLRLDLEMILNGINNYKNPKLTRMLTAKFYMPKKATELKHLQCLE
EALKPLEEVLNLAQSKNFHFDPRDVVSNINVFVLELKGSETTFMCEYADETATIVEFLNRW
ITFCTSIISTLT

In some embodiments, the subject IL-2 mutein having a greater binding affinity for IL-2Rβ as compared to wild-type human IL-2, includes the amino acid substitutions L18R, Q22E, L80F, R81D, L85V, I86V, I92F, Q126T, and F42A. In some embodiments, the IL-2 mutein has the amino acid sequence:

(SEQ ID NO: 7)
APTSSSTKKTQLQLEHLRLDLEMILNGINNYKNPKLTRMLTAKFYMPKKATELKHLQCLE
EELKPLEEVLNLAQSKNFHFDPRDVVSNINVFVLELKGSETTFMCEYADETATIVEFLNRW
ITFCTSIISTLT

In some embodiments, the IL-2 mutein sequence is at least about 90% identical to any one of SEQ ID NO: 1 through SEQ ID NO: 7. In some embodiments, the IL-2 mutein sequence is at least about 95% identical to any one of SEQ ID NO: 1 through SEQ ID NO: 7. In some embodiments, the IL-2 mutein sequence is at least about 98% identical to any one of SEQ ID NO: 1 through SEQ ID NO: 7. In some embodiments, the IL-2 mutein sequence is at least about 99% identical to any one of SEQ ID NO: 1 through SEQ ID NO: 7.

### B. IL-2 Mutein Fusion Proteins

The IL-2 muteins can be prepared as fusion or chimeric polypeptides that include a subject IL-2 mutein and a heterologous polypeptide (i.e., a polypeptide that is not IL-2 or a mutant thereof) (see, e.g., U.S. Pat. No. 6,451,308). Exemplary heterologous polypeptides can increase the circulating half-life of the chimeric polypeptide in vivo, and may, therefore, further enhance the properties of the mutant IL-2 polypeptides. In various embodiments, the polypeptide that increases the circulating half-life may be a serum albumin, such as human serum albumin, PEG, PEG-derivatives, or the Fc region of the IgG subclass of antibodies that lacks the IgG heavy chain variable region. Exemplary Fc regions can include a mutation that inhibits complement fixation and Fc receptor binding, or it may be lytic, i.e., able to bind complement or to lyse cells via another mechanism, such as antibody-dependent complement lysis (ADCC; U.S. Ser. No. 08/355,502 filed Dec. 12, 1994).

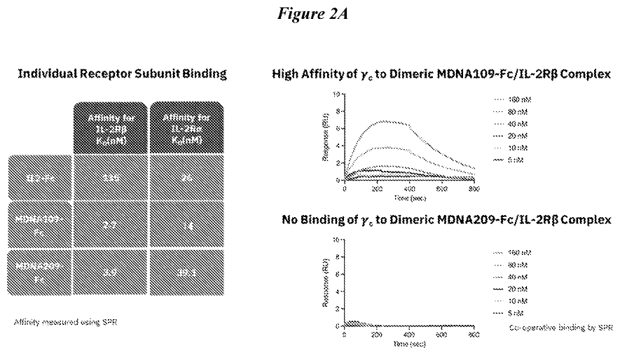

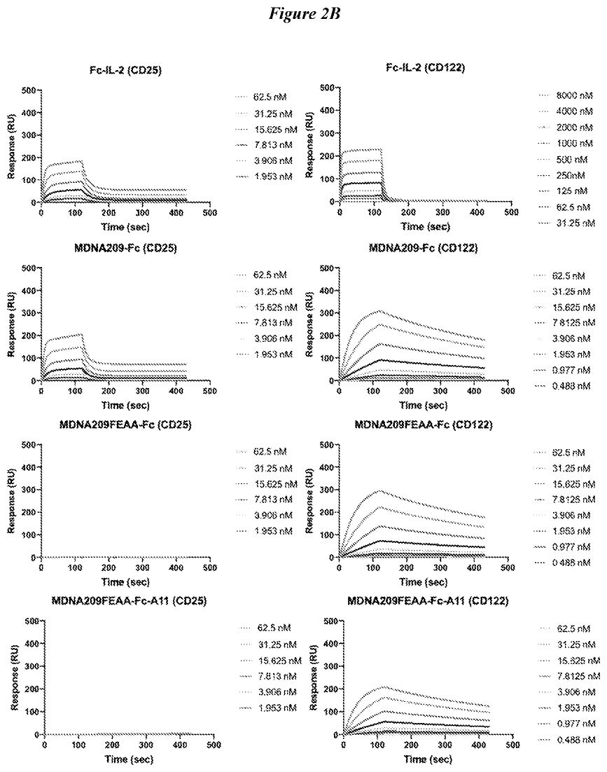

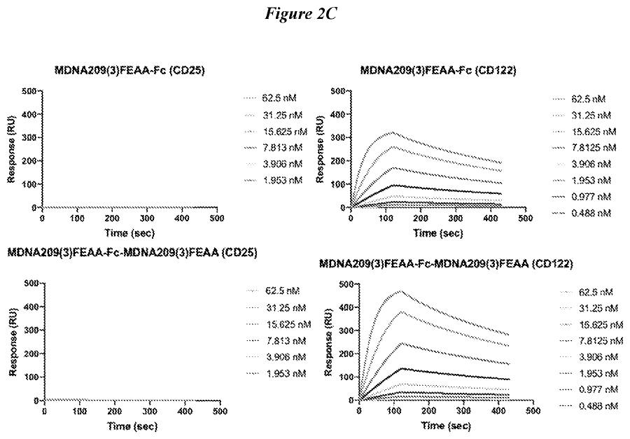

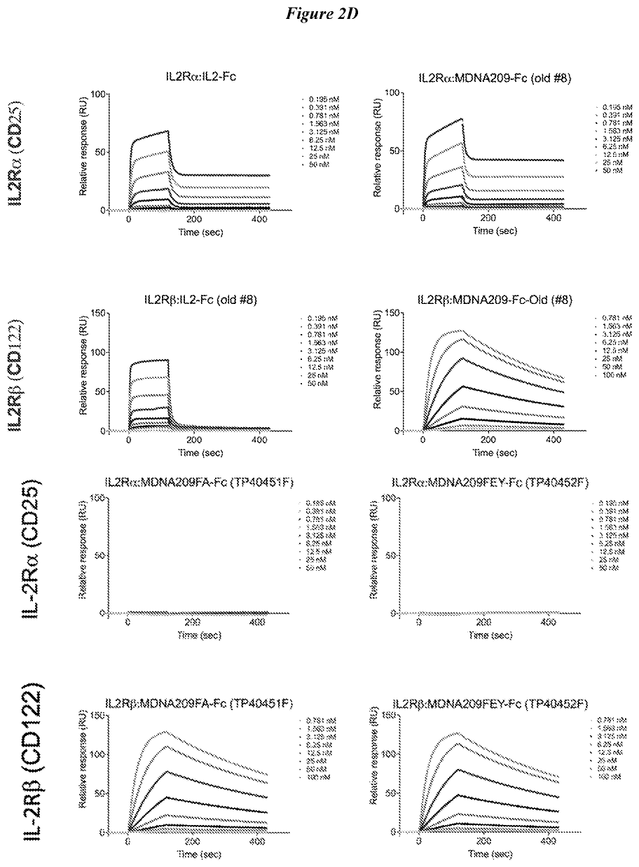

The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. In some embodiments, the IL-2 mutein fusion protein (e.g., an IL-2 mutein as described herein) includes an IgG1, IgG2, IgG3, or IgG4 Fc region (see, for example, sequences in FIG. 2A-2B). In some embodiments, the Fc region comprises the substitution N297A.

In some embodiments, the IL-2 mutein is linked directly or indirectly to the heterologous fusion polypeptide.

In some embodiments, the IL-2 mutein is linked directly to the Fc region. In some embodiments, the IL-2 mutein is linked to the Fc region via a linker peptide, such as GGGGS. In some embodiments, the linker is (GGGGS)n, wherein n is an integer between 1 and 10. In some embodiments, the linker is GGGGS. In some embodiments, the linker is GGGGSGGGGS (SEQ ID NO: 14). In some embodiments, the linker is GGGGSGGGGGGGGS (SEQ ID NO: 15). In some embodiments, the linker is GGGGSGGGGSGGGGSGGGGS (SEQ ID NO: 16). In some embodiments, the linker is GGGGSGGGGSGGGGSGGGGS (SEQ ID NO: 17).

The Fc region can be “lytic” or “non-lytic,” but is typically non-lytic. A non-lytic Fc region typically lacks a high affinity Fc receptor binding site and a C′1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2:125, 1994).

In other embodiments, the chimeric polypeptide can include a subject IL-2 mutein and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

In other embodiments, the chimeric polypeptide includes the mutant IL-2 polypeptide and a heterologous polypeptide that functions to enhance expression or direct cellular localization of the mutant IL-2 polypeptide, such as the Aga2p agglutinin subunit (see, e.g., Boder and Wittrup, Nature Biotechnol. 15:553-7, 1997).

In other embodiments, a chimeric polypeptide including a mutant IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In other embodiments, a chimeric polypeptide including a mutant IL-2 and an IL-4 protein can be generated. Any IL-4 sequence or variant thereof can be used in a fusion with an IL-2 mutant or variant, including those as described herein. In some embodiments, a mutant IL-2 is fused to an IL-4 mutant having a sequence of SEQ ID NO: 23, as shown below:

(KFR)
SEQ ID NO: 23
KCDITLQEIIKTLNSLTEQKTLCTELTVTDIFAASKNTTEKETFCRAATVLRQFYSHHEKDTR
CLGATAQQFHRHKQLIRFLKRLDRNLWGLAGLNSCPVKEANQSTLENFLERLKTIMKEKF
RKCSS

In some embodiments, a mutant IL-2 is fused to an IL-4 mutant having a sequence of SEQ ID NO: 24, as shown below:

(cpRGA)
SEQ ID NO: 24
MDTTEKETFCRAATVLRQFYSHHEKDTRCLGATAQQFHRHKQLIRFLKRLDRNLWGLAG
LNSCPVKEANQSTLENFLERLRVIMQSKWFKCGAGGNGGHKCDITLQEIIKTLNSLTEQKT
LCTELTVTDIFAAS

In some embodiments, a mutant IL-2 is fused to an IL-4 mutant having a sequence of SEQ ID NO: 25, as shown below:

(RGA)
SEQ ID NO: 25
HKCDITLQEIIKTLNSLTEQKTLCTELTVTDIFAASKNTTEKETFCRAATVLRQFYSHHEKDT
RCLGATAQQFHRHKQLIRFLKRLDRNLWGLAGLNSCPVKEANQSTLENFLERLRVIMQSK
WFKCGA

In some embodiments, a mutant IL-2 is fused to an IL-4 mutant having a sequence that is at least about 90% identical to SEQ ID NO: 23. In some embodiments, a mutant IL-2 is fused to an IL-4 mutant having a sequence that is at least about 90% identical to SEQ ID NO: 24. In some embodiments, a mutant IL-2 is fused to an IL-4 mutant having a sequence that is at least about 90% identical to SEQ ID NO: 25. In some embodiments, a mutant IL-2 is fused to an IL-4 mutant having a sequence that is at least about 95% identical to SEQ ID NO: 23. In some embodiments, a mutant IL-2 is fused to an IL-4 mutant having a sequence that is at least about 95% identical to SEQ ID NO: 24. In some embodiments, a mutant IL-2 is fused to an IL-4 mutant having a sequence that is at least about 95% identical to SEQ ID NO: 25. In some embodiments, a mutant IL-2 is fused to an IL-4 mutant having a sequence that is at least about 98% identical to SEQ ID NO: 23. In some embodiments, a mutant IL-2 is fused to an IL-4 mutant having a sequence that is at least about 98% identical to SEQ ID NO: 24. In some embodiments, a mutant IL-2 is fused to an IL-4 mutant having a sequence that is at least about 98% identical to SEQ ID NO: 25. In some embodiments, a mutant IL-2 is fused to an IL-4 mutant having a sequence that is at least about 99% identical to SEQ ID NO: 23. In some embodiments, a mutant IL-2 is fused to an IL-4 mutant having a sequence that is at least about 99% identical to SEQ ID NO: 24. In some embodiments, a mutant IL-2 is fused to an IL-4 mutant having a sequence that is at least about 99% identical to SEQ ID NO: 25.

In some embodiments, SEQ ID NO: 23 is linked to an IL-2 or IL-2 mutein as described herein. In some embodiments, SEQ ID NO: 24 is linked to an IL-2 or IL-2 mutein as described herein. In some embodiments, SEQ ID NO: 25 is linked to an IL-2 or IL-2 mutein as described herein.

In some embodiments, the IL-2 mutein sequence is at least about 90% identical to any one of SEQ ID NO: 1 through SEQ ID NO: 7 (for example, any of the IL-2 sequences provided herein). In some embodiments, the IL-2 mutein sequence is at least about 95% identical to any one of SEQ ID NO: 1 through SEQ ID NO: 7 (for example, any of the IL-2 sequences provided herein). In some embodiments, the IL-2 mutein sequence is at least about 98% identical to any one of SEQ ID NO: 1 through SEQ ID NO: 7 (for example, any of the IL-2 sequences provided herein). In some embodiments, the IL-2 mutein sequence is at least about 99% identical to any one of SEQ ID NO: 1 through SEQ ID NO: 7 (for example, any of the IL-2 sequences provided herein). In some embodiments, the IL-2 mutein further comprises and/or is conjugated to an A11 mutein or variant thereof, including for example Fc-A11 (1:2); version 1 (SEQ ID NO: 9) or Fc-A11 (1:2); version 2 (SEQ ID NO: 10).

In some embodiments, the IL-2 mutant fusion protein sequence is at least about 90% identical to any one of SEQ ID NO: 18 through SEQ ID NO: 22. In some embodiments, the IL-2 mutant fusion protein sequence is at least about 95% identical to any one of SEQ ID NO: 18 through SEQ ID NO: 22. In some embodiments, the IL-2 mutant fusion protein sequence is at least about 98% identical to any one of SEQ ID NO: 18 through SEQ ID NO: 22. In some embodiments, the IL-2 mutant fusion protein sequence is at least about 99% identical to any one of SEQ ID NO: 18 through SEQ ID NO: 22. In some embodiments, the IL-2 mutant fusion protein sequence comprises any one of SEQ ID NO: 18 through SEQ ID NO: 22.

TABLE 3
List of Exemplary MDNA209 Fusions

| SEQ ID NO: | |
|---|---|
| (Information) | Amino acid sequence |
| SEQ ID NO: 18 MDNA209-Albumin Mutations: L18R, Q22E, L80F, R81D, L85V, I86V, I92F, Q126T, S130R | 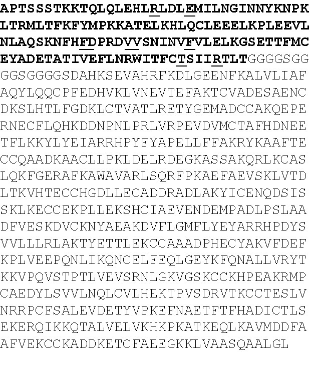 |
| SEQ ID NO: 19 MDNA209-Fc-KFR (2:1:2) Mutations: MDNA209: L18R, Q22E, L80F, R81D, L85V, I86V, I92F, Q126T, S13OR KFR: R121K, Y124F, S125R | 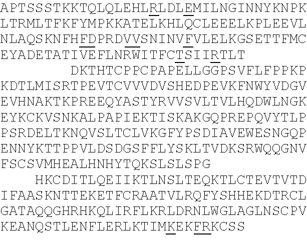 |
| SEQ ID NO: 20 MDNA209FEAA-Fc-KFR (2:1:2) Mutations: 209FEAA: L18R, Q22E, L80F, R81D, L85V, I86V, I92F, Q126T, S130R, F42A, E62A KFR: R121K, Y124F, S125R | 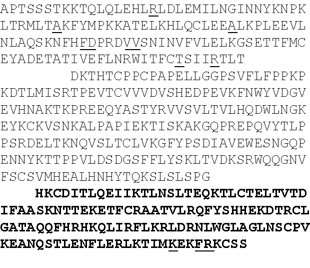 |
| SEQ ID NO: 21 MDNA209-Fc-RGA (2:1:2) Mutations: MDNA209: L18R, Q22E, L80F, R81D, L85V, I86V, I92F, Q126T, S130R RGA: K117R, T118V, R121Q, E122S, Y124W, S125F, S128G, S129A | 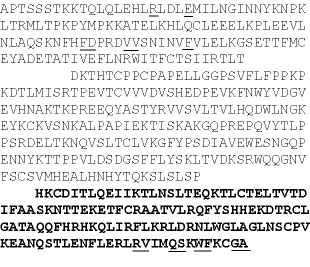 |
| SEQ ID NO: 22 MDNA209FEAA-Fc-RGA (2:1:2) Mutations: MDNA209FEAA: L18R, Q22E, L80F, R81D, L85V, I86V, I92F, Q126T, S130R, F42A, E62A RGA: K117R, T118V, R121Q, E122S, Y124W, S125F, S128G, S129A | 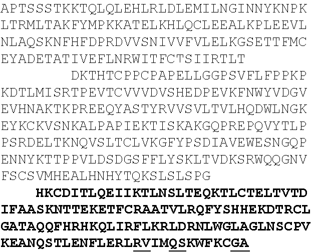 |

### C. Recombinant Expression of IL-2 Muteins, Expression Vectors and Host Cells

In various embodiments, polypeptides used in the practice of the instant invention are synthetic, or are produced by expression of a recombinant nucleic acid molecule. In the event the polypeptide is a chimera (e.g., a fusion protein containing at least a mutant IL-2 polypeptide and a heterologous polypeptide), it can be encoded by a hybrid nucleic acid molecule containing one sequence that encodes all or part of the IL-2 mutein, and a second sequence that encodes all or part of the heterologous polypeptide. For example, subject IL-2 muteins described herein may be fused to a hexa-histidine tag to facilitate purification of bacterially expressed protein, or to a hemagglutinin tag to facilitate purification of protein expressed in eukaryotic cells.

Methods for constructing a DNA sequence encoding the IL-2 muteins and expressing those sequences in a suitably transformed host include, but are not limited to, using a PCR-assisted mutagenesis technique. Mutations that consist of deletions or additions of amino acid residues to an IL-2 polypeptide can also be made with standard recombinant techniques. In the event of a deletion or addition, the nucleic acid molecule encoding IL-2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

The complete amino acid sequence can be used to construct a back-translated gene. A DNA oligomer containing a nucleotide sequence coding for IL-2 mutein can be synthesized. For example, several small oligonucleotides coding for portions of the desired polypeptide can be synthesized and then ligated. The individual oligonucleotides typically contain 5′ or 3′ overhangs for complementary assembly.

In addition to generating mutant polypeptides via expression of nucleic acid molecules that have been altered by recombinant molecular biological techniques, subject IL-2 muteins can be chemically synthesized. Chemically synthesized polypeptides are routinely generated by those of skill in the art.

Once assembled (by synthesis, site-directed mutagenesis or another method), the DNA sequences encoding an IL-2 mutein will be inserted into an expression vector and operatively linked to an expression control sequence appropriate for expression of the IL-2 mutein in the desired transformed host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

The DNA sequence encoding the IL-2 mutein, whether prepared by site directed mutagenesis, chemical synthesis or other methods, can also include DNA sequences that encode a signal sequence. Such signal sequence, if present, should be one recognized by the cell chosen for expression of the IL-2 mutein. It can be prokaryotic, eukaryotic or a combination of the two. It can also be the signal sequence of native IL-2. The inclusion of a signal sequence depends on whether it is desired to secrete the IL-2 mutein from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild-type IL-2 signal sequence be used.

### D. Nucleic Acid Molecules Encoding Mutant IL-2

In some embodiments the subject IL-2 mutein, either alone or as a part of a chimeric polypeptide, such as those described above, can be obtained by expression of a nucleic acid molecule. Just as IL-2 muteins can be described in terms of their identity with wild-type IL-2 polypeptides, the nucleic acid molecules encoding them will necessarily have a certain identity with those that encode wild-type IL-2. For example, the nucleic acid molecule encoding a subject IL-2 mutein can be at least 50%, at least 65%, preferably at least 75%, more preferably at least 85%, and most preferably at least 95% (e.g., 99%) identical to the nucleic acid encoding wild-type IL-2 (e.g., SEQ ID NO: 8).

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a mutant IL-2) is incorporated into a vector (e.g., a plasmid or viral vector) or into the genome of a heterologous cell (or the genome of a homologous cell, at a position other than the natural chromosomal location).

As described above, the subject IL-2 mutein may exist as a part of a chimeric polypeptide. In addition to, or in place of, the heterologous polypeptides described above, a subject nucleic acid molecule can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-hosphotransferase (HPH), thymidine kinase (TK), lacz (encoding β-galactosidase), and xanthine guanine phosphoribosyltransferase (XGPRT). One of skill in the art will be aware of additional useful reagents, for example, of additional sequences that can serve the function of a marker or reporter.

The subject nucleic acid molecules can be obtained by introducing a mutation into IL-2-encoding DNA obtained from any biological cell, such as the cell of a mammal. Thus, the subject nucleic acids (and the polypeptides they encode) can be those of a mouse, rat, guinea pig, cow, sheep, horse, pig, rabbit, monkey, baboon, dog, or cat. In one embodiment, the nucleic acid molecules will be those of a human.

### E. Expression of Mutant IL-2 Gene Products

The nucleic acid molecules described above can be contained within a vector that is capable of directing their expression in, for example, a cell that has been transduced with the vector. Accordingly, in addition to the subject IL-2 muteins, expression vectors containing a nucleic acid molecule encoding a subject IL-2 mutein and cells transfected with these vectors are among the preferred embodiments.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the IL-2 muteins to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction of a Modular Dihydrafolate Reductase cDNA Gene: Analysis of Signals Utilized for Efficient Expression”, Mol. Cell. Biol., 2, pp. 1304-19 (1982)) or glutamine synthetase (“GS”) amplification (see, e.g., U.S. Pat. No. 5,122,464 and European published application 338,841).

In some embodiments, the human IL-2 muteins of the present disclosure will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are included also.

Exemplary recombinant expression vectors can include one or more regulatory sequences, selected on the basis of the host cells to be used for expression, operably linked to the nucleic acid sequence to be expressed.

The expression constructs or vectors can be designed for expression of an IL-2 mutein or variant thereof in prokaryotic or eukaryotic host cells.

Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

Expression of proteins in prokaryotes is most often carried out in Escherichia coli with vectors containing constitutive or inducible promoters. Strategies to maximize recombinant protein expression in E. coli can be found, for example, in Gottesman (1990) in Gene Expression Technology: Methods in Enzymology 185 (Academic Press, San Diego, Calif.), pp. 119-128 and Wada et al. (1992) Nucleic Acids Res. 20:2111-2118. Processes for growing, harvesting, disrupting, or extracting the IL-2 mutein or variant thereof from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

In some embodiments the recombinant IL-2 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2^{nd} ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-2 muteins of the present disclosure can be optimized for expression in the host cell of interest. The G-C content of the sequence can be adjusted to levels average for a given cellular host, as calculated by reference to known genes expressed in the host cell. Methods for codon optimization are well known in the art. Codons within the IL-2 mutein coding sequence can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif.) for use in insect cells.

In some embodiments nucleic acid inserts, which encode the subject IL-2 muteins in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject IL-2 mutein, particularly as regards potential secondary structures. Hosts should be selected by consideration of their compatibility with the chosen vector, the toxicity of the product coded for by the DNA sequences of this invention, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Useful expression vectors for eukaryotic hosts, include, for example, vectors with expression control sequences from SV40, bovine papilloma virus, adenovirus and cytomegalovirus. Useful expression vectors for bacterial hosts include known bacterial plasmids, such as plasmids from E. coli, including col E1, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof. Useful vectors for insect cells include pVL 941 and pFastBac™ 1 (GibcoBRL, Gaithersburg, Md.). Cate et al., “Isolation Of The Bovine And Human Genes For Mullerian Inhibiting Substance And Expression Of The Human Gene In Animal Cells”, Cell, 45, pp. 685-98 (1986).

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject IL-2 mutein disclosed herein are also features of the invention. A cell of the invention is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the invention.

The precise components of the expression system are not critical. For example, an IL-2 mutein can be produced in a prokaryotic host, such as the bacterium E. coli, or in a eukaryotic host, such as an insect cell (e.g., an Sf21 cell), or mammalian cells (e.g., CHO, HEK293, COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, IL-2 muteins obtained will be glycosylated or unglycosylated depending on the host organism used to produce the mutein. If bacteria are chosen as the host then the IL-2 mutein produced will be unglycosylated. Eukaryotic cells, on the other hand, will glycosylate the IL-2 muteins, although perhaps not in the same way as native-IL-2 is glycosylated. The IL-2 mutein produced by the transformed host can be purified according to any suitable method. Various methods are known for purifying IL-2. See, e.g. Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc. IL-2 muteins can be isolated from inclusion bodies generated in E. coli, or from conditioned medium from either mammalian or yeast cultures producing a given mutein using cation exchange, gel filtration, and/or reverse phase liquid chromatography.

Another exemplary method of constructing a DNA sequence encoding the IL-2 muteins is by chemical synthesis. This includes direct synthesis of a peptide by chemical means of the protein sequence encoding for an IL-2 mutein exhibiting the properties described. This method can incorporate both natural and unnatural amino acids at positions that affect the interactions of IL-2 with the IL-2Rα, the IL-2Rβ and/or the IL-2Rγ. Alternatively a gene which encodes the desired IL-2 mutein can be synthesized by chemical means using an oligonucleotide synthesizer. Such oligonucleotides are designed based on the amino acid sequence of the desired IL-2 mutein, and preferably selecting those codons that are favored in the host cell in which the recombinant mutein will be produced. In this regard, it is well recognized that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated that for a given DNA sequence encoding a particular IL-2 mutein, there will be many DNA degenerate sequences that will code for that IL-2 mutein. For example, it will be appreciated that in addition to the preferred DNA sequence for mutein H9, there will be many degenerate DNA sequences that code for the IL-2 mutein shown. These degenerate DNA sequences are considered within the scope of this disclosure. Therefore, “degenerate variants thereof” in the context of this invention means all DNA sequences that code for and thereby enable expression of a particular mutein.

The biological activity of the IL-2 muteins can be assayed by any suitable method known in the art. Such assays include PHA-blast proliferation and NK cell proliferation.

### F. Methods of Treatment

In some embodiments, subject IL-2 muteins, and/or nucleic acids expressing them, can be administered to a subject to treat a disorder associated with abnormal apoptosis or a differentiative process (e.g., cellular proliferative disorders or cellular differentiative disorders, such as cancer, by, for example, producing an active or passive immunity). In the treatment of such diseases, the disclosed IL-2 muteins may possess advantageous properties, such as reduced vascular leak syndrome. In some embodiments, the IL-2 mutein is any IL-2 mutein or variant disclosed herein. In some embodiments, the IL-2 mutein sequence is at least about 90% identical to any one of SEQ ID NO: 1 through SEQ ID NO: 7. In some embodiments, the substitutions in the IL-2 mutein are numbered in accordance with wild-type human IL-2 of SEQ ID NO: 8. In some embodiments, the IL-2 mutein is a fusion protein. In some embodiments, the IL-2 mutein is associated with and/or expressed by a CAR-T construct. In some embodiments, the IL-2 mutein is expressed by and/or associated with an oncolytic virus.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate cancer, ovarian cancer, breast cancer, endometrial cancer, multiple myeloma, melanoma, lymphomas, lung cancers including small cell lung cancer, kidney cancer, liver cancer, colon cancer, colorectal cancer, pancreatic cancer, gastric cancer, and brain cancer.

The mutant IL-2 polypeptides can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues.

Additional examples of proliferative disorders include hematopoietic neoplastic disorders.

Alternatively, or in addition to methods of direct administration to patients, in some embodiments, mutant IL-2 polypeptides can be used in ex vivo methods. For example, cells (e.g., peripheral blood lymphocytes or purified populations of lymphocytes isolated from a patient and placed or maintained in culture) can be cultured in vitro in culture medium and the contacting step can be affected by adding the IL-2 mutant to the culture medium. The culture step can include further steps in which the cells are stimulated or treated with other agents, e.g., to stimulate proliferation, or to expand a population of cells that is reactive to an antigen of interest (e.g., a cancer antigen or a viral antigen). The cells are then administered to the patient after they have been treated.

In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination with nivolumab for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination BMS-936558 for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination MDX-1106 for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination ONO-4538 for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination AMP224 for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination CT-011 for the treatment of cancer. In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination MK-3475 for the treatment of cancer. In some embodiments, the IL-2 mutein comprises substitutions L18R, Q22E, L80F, R81D, L85V, I86V, I92F, and Q126T. In some embodiments, the IL-2 mutein further comprises a substitution at position S130R. In some embodiments, the IL-2 mutein further comprises a substitution at position S130R. In some embodiments, the IL-2 mutein further comprises a substitution at position F42A. In some embodiments, the IL-2 mutein further comprises a substitution at position E62A. In some embodiments, the IL-2 mutein further comprises a substitution at position Y45A. In some embodiments, the substitutions in the IL-2 mutein comprise L18R, Q22E, and Q126T, and further comprise a group of amino acid substitutions selected from the group consisting of F42A, Y45A, E62A, L80F, R81D, L85V, I86V, I92F, Q126T, and S130R, numbered in accordance with wild-type human IL-2 of SEQ ID NO: 8. In some embodiments, the IL-2 mutein comprises substitutions L18R, Q22E, L80F, R81D, L85V, I86V, I92F, Q126T, and S130R, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, F42A, and E62A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, and F42A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, S130R, F42A, and Y45A, numbered in accordance with wild-type human IL-2 of SEQ ID NO: 8. In some embodiments, the IL-2 mutein comprises substitutions L18R, Q22E, L80F, R81D, L85V, I86V, I92F, and Q126T, numbered in accordance with wild-type human IL-2 of SEQ ID NO: 8. In some embodiments, the IL-2 mutein comprises substitutions L18R, Q22E, L80F, R81D, L85V, I86V, I92F, Q126T, F42A, and E62A, numbered in accordance with wild-type human IL-2 of SEQ ID NO: 8. In some embodiments, the IL-2 mutein comprises substitutions L18R, Q22E, L80F, R81D, L85V, I86V, I92F, Q126T, and F42A, numbered in accordance with wild-type human IL-2 of SEQ ID NO: 8.

In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination with an antibody and/or immunotherapy including but not limited to, anti-CTLA4 mAbs, such as ipilimumab, tremelimumab; anti-PD-L1 antagonistic antibodies such as BMS-936559/MDX-1105, MEDI4736, RG-7446/MPDL3280A; anti-LAG-3 such as IMP-321; agonistic antibodies targeting immunostimulatory proteins, including anti-CD40 mAbs such as CP-870,893, lucatumumab, dacetuzumab; anti-CD137 mAbs (anti-4-1-BB antibodies) such as BMS-663513 urelumab (anti-4-1BB antibody; see, for example, U.S. Pat. Nos. 7,288,638 and 8,962,804, incorporated by reference herein in their entireties); lirilumab (anti-KIR mAB; IPH2102/BMS-986015; blocks NK cell inhibitory receptors) and PF-05082566 (utomilumab; see, for example, U.S. Pat. Nos. 8,821,867; 8,337,850; and 9,468,678, as well as International Patent Application Publication No. WO 2012/032433, incorporated by reference herein in their entireties); anti-OX40 mAbs (see, for example, WO 2006/029879 or WO 2010/096418, incorporated by reference herein in their entireties); anti-GITR mAbs such as TRX518 (see, for example, U.S. Pat. No. 7,812,135, incorporated by reference herein in its entirety); anti-CD27 mAbs, such as varlilumab CDX-1127 (see, for example, WO 2016/145085 and U.S. Patent Publication Nos. US 2011/0274685 and US 2012/0213771, incorporated by reference herein in their entireties) anti-ICOS mAbs (for example, MEDI-570, JTX-2011, and anti-TIM-3 antibodies (see, for example, WO 2013/006490 or U.S. Patent Publication No US 2016/0257758, incorporated by reference herein in their entireties) for the treatment of cancer.

In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination with another antibody which can include monoclonal antibodies to prostate cancer, ovarian cancer, breast cancer, endometrial cancer, multiple myeloma, melanoma, lymphomas, lung cancers including small cell lung cancer, kidney cancer, colorectal cancer, pancreatic cancer, gastric cancer, brain cancer (see, generally www.clinicaltrials.gov), for the treatment of cancer.

In some embodiments, the IL-2 mutein comprising substitutions L18R, Q22E, and Q126T, and one or more substitutions selected from the group consisting of F42A, Y45A, E62A, L80F, R81D, L85V, I86V, I92F, Q126T, and S130R, numbered in accordance with human wild-type IL-2 (SEQ ID NO: 8) is used in combination with antibodies for antibody-dependent cell-mediated cytotoxicity (ADCC) for the treatment of cancer.

### G. Pharmaceutical Compositions and Methods of Administration

In some embodiments, subject IL-2 muteins and nucleic acids can be incorporated into compositions, including pharmaceutical compositions. Such compositions typically include the polypeptide or nucleic acid molecule and a pharmaceutically acceptable carrier.

A pharmaceutical composition is formulated to be compatible with its intended route of administration. The mutant IL-2 polypeptides of the invention may be given orally, but it is more likely that they will be administered through a parenteral route, including for example intravenous administration. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation IL-2 muteins, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the IL-2 muteins or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, compounds (mutant IL-2 polypeptides or nucleic acids) can also be prepared in the form of suppositories (e.g., with conventional suppository bases such as cocoa butter and other glycerides) or retention enemas for rectal delivery.

In some embodiments, compounds (subject IL-2 muteins or nucleic acids) can also be administered by transfection or infection using methods known in the art, including but not limited to the methods described in McCaffrey et al. (Nature 418:6893, 2002), Xia et al. (Nature Biotechnol. 20:1006-1010, 2002), or Putnam (Am. J. Health Syst. Pharm. 53:151-160, 1996, erratum at Am. J. Health Syst. Pharm. 53:325, 1996).

In one embodiment, the IL-2 muteins or nucleic acids are prepared with carriers that will protect the mutant IL-2 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of IL-2 muteins, or nucleic acids compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a subject IL-2 mutein (i.e., an effective dosage) depends on the polypeptide or antibody selected. In some embodiments, single dose amounts of the IL-2 mutein can be in the range of approximately 0.001 mg/kg to 0.1 mg/kg of patient body weight can be administered. In some embodiments, doses of the IL-2 mutein of about 0.005 mg/kg, 0.01 mg/kg, 0.025 mg/kg, 0.05 mg/kg, 0.1 mg/kg, 0.25 mg/kg, 0.5 mg/kg, 1.0 mg/kg, 5.0 mg/kg, 10.0 mg/kg may be administered. In some embodiments, 600,000 IU/kg is administered (IU can be determined by a lymphocyte proliferation bioassay and is expressed in International Units (IU) as established by the World Health Organization 1^{st} International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject IL-2 muteins can include a single treatment or, can include a series of treatments. In one embodiment, the compositions are administered every 8 hours for five days, followed by a rest period of 2 to 14 days, e.g., 9 days, followed by an additional five days of administration every 8 hours. In some embodiments, administration is 3 doses administered every 4 days.

The pharmaceutical compositions can be included in a container, pack, or dispenser together with instructions for administration.

The following examples are provided to describe certain embodiments of the invention provided herein and are not to be construed to as limiting.

### Examples

### Example 1: Analysis of IL-2 Antagonists

### Binding-Surface Plasmon Resonance (SPR) Methodology

Surface Plasmon Resonance (SPR) technology enables label-free, real-time biomolecular interaction analysis. Binding interactions between proteins using purified preparations or complex mixtures can be examined quantitatively in their native state and can assess binding specificity, stoichiometry, concentration, thermodynamics, kinetics (association, kₐ, and dissociation, k_{d}, rate constants), and overall affinity (equilibrium dissociation constant, K_{D}=k_{d}/kₐ). Sensor chips (CM5) were activated (420 s at a flow rate of 10 μL/min) and 50 μg/mL of Anti-histidine antibody (in Immobilization buffer 10 mM Sodium Acetate (pH4.5)) was injected (420 s at a flow rate of 10 μL/min). For ligand capturing, samples were diluted to 5 μg/mL (HEPES, with 0.005% Tween-20, ph7.4) and injected (flow rate of 10 μL/min) to reach a capture level of ~200 RU. Receptors were diluted and up to 8 concentrations as indicated and injected (flow rate of 30 μL/min) for an association phase of 120 seconds, followed by 300 seconds dissociation. Constructs examined by SPR are shown in Tables 4 and 5.

TABLE 4
Constructs examined in SPR

| Reagent | | |
|---|---|---|
| IL-2 | Source | Lot |
| MDNA209-Fc (2:1) | Abzena | Construct #8 |
| MDNA209FEAA-Fc (2:1) | Lake Pharma | TP36871F |
| MDNA209FA-Fc (2:1) | Lake Pharma | TP40451F |
| MDNA209FEY-Fc (2:1) | Lake Pharma | TP40452F |
| MDNA209FEAA-Fc-A11 (2:1:2) | Abzena | Construct #10 |
| MDNA209(3)FEAA-Fc (2:1) | Lake Pharma | TP36872F |
| MDNA209(3)FEAA-Fc- | Lake Pharma | TP36873F |
| MDNA209(3)FEAA (2:1:2) | | |

TABLE 5
List of MDNA209 Variants analyzed

| SEQ ID NO: | |
|---|---|
| (Information) | Amino acid sequence |
| SEQ ID NO: 1 | APTSSSTKKTQLQLEHLRLDLEMILNGINNYKNP |
| (MDNA209; Mutations: L18R, Q22E, | KLTRMLTFKFYMPKKATELKHLQCLEEELKPLEE |
| L80F, R81D, L85V, I86V, 192F, | VLNLAQSKNFHFDPRDVVSNINVFVLELKGSETT |
| Q126T, S130R) | FMCEYADETATIVEFLNRWITFCTSIIRTLT |
| SEQ ID NO: 2 | APTSSSTKKTQLQLEHLRLDLEMILNGINNYKNPK |
| (MDNA209FEAA; Mutations: L18R, | LTRMLTAKFYMPKKATELKHLQCLEEALKPLEEVL |
| Q22E, L80F, R81D, L85V, I86V, 192F, | NLAQSKNFHFDPRDVVSNINVFVLELKGSETTFMC |
| Q126T, S130R, F42A, E62A) | EYADETATIVEFLNRWITFCTSIIRTLT |
| SEQ ID NO: 3 | APTSSSTKKTQLQLEHLRLDLEMILNGINNYKNPK |
| (MDNA209FA; Mutations: L18R, | LTRMLTAKFYMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T, S130R, F42A) | EYADETATIVEFLNRWITFCTSIIRTLT |
| SEQ ID NO: 4 | APTSSSTKKTQLQLEHLRLDLEMILNGINNYKNPK |
| (MDNA209FEY; Mutations: L18R, | LTRMLTAKFAMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T, S130R, F42A, Y45A) | EYADETATIVEFLNRWITFCTSIIRTLT |
| SEQ ID NO: 5 | APTSSSTKKTQLQLEHLRLDLEMILNGINNYKNPK |
| (MDNA209(3); Mutations: L18R, | LTRMLTFKFYMPKKATELKHLQCLEEELKPLEEVL |
| Q22E, L80F, R81D, L85V, I86V, 192F, | NLAQSKNFHFDPRDVVSNINVFVLELKGSETTFMC |
| Q126T) | EYADETATIVEFLNRWITFCTSIISTLT |
| SEQ ID NO: 6 | APTSSSTKKTQLQLEHLRLDLEMILNGINNYKNPK |
| (MDNA209(3)FEAA; Mutations: | LTRMLTAKFYMPKKATELKHLQCLEEALKPLEEVL |
| L18R, Q22E, L80F, R81D, L85V, | NLAQSKNFHFDPRDVVSNINVFVLELKGSETTFMC |
| I86V, I92F, Q126T, F42A, E62A) | EYADETATIVEFLNRWITFCTSIISTLT |
| SEQ ID NO: 7 | APTSSSTKKTQLQLEHLRLDLEMILNGINNYKNPK |
| (MDNA209(3)FA; Mutations: L18R, | LTRMLTAKFYMPKKATELKHLQCLEEELKPLEEVL |
| Q22E, L80F, R81D, L85V, I86V, I92F, | NLAQSKNFHFDPRDVVSNINVFVLELKGSETTFMC |
| Q126T, F42A) | EYADETATIVEFLNRWITFCTSIISTLT |
| SEQ ID NO: 8 | APTSSSTKKTQLQLEHLLLDLQMILNGINNYKNPK |
| (human wild-type IL-2) | LTRMLTFKFYMPKKATELKHLQCLEEELKPLEEVL |
| | NLAQSKNFHLRPRDLISNINVIVLELKGSETTFMC |
| | EYADETATIVEFLNRWITFCQSIISTLT |
| SEQ ID NO: 9 | Gene 1 and Gene 2: |
| (Fc-A11 (1:2); version 1) | DKTHTCPPCPAPELLGGPSVFLFPPKPKDTLMISR |
| | TPEVTCVVVDVSHEDPEVKFNWYVDGVEVHNAKTK |
| | PREEQYASTYRVVSVLTVLHQDWLNGKEYKCKVSN |
| | KALPAPIEKTISKAKGQPREPQVYTLPPSRDELTK |
| | NQVSLTCLVKGFYPSDIAVEWESNGQPENNYKTTP |
| | PVLDSDGSFFLYSKLTVDKSRWQQGNVFSCSVMHE |
| | ALHNHYTQKSLSLSPGGGGGSGGGGSGGGGSPGPV |
| | PPSTAVRELIEELINITQNQKAPLCNGSMVWSINR |
| | TAGMYCAALESLINVSGCSAIEKTQRMLSGFCPHK |
| | VSAGQFSSLHVRSSKIEVAQFVKDLLFHLRTLFRE |
| | GQFN |
| SEQ ID NO: 10 | Gene 1 and Gene 2: |
| (Fc-A11 (1:2); version 2) | DKTHTCPPCPAPELLGGPSVFLFPPKPKDTLMISR |
| | TPEVTCVVVDVSHEDPEVKFNWYVDGVEVHNAKTK |
| | PREEQYASTYRVVSVLTVLHQDWLNGKEYKCKVSN |
| | KALPAPIEKTISKAKGQPREPQVYTLPPSRDELTK |
| | NQVSLTCLVKGFYPSDIAVEWESNGQPENNYKTTP |
| | PVLDSDGSFFLYSKLTVDKSRWQQGNVFSCSVMHE |
| | ALHNHYTQKSLSLSPGGGGGSGGGGSGGGGSPGPV |
| | PPSTAVRELIEELINITQNQKAPLCNGSMVWSINL |
| | TAGMYCAALESLINVSGCSAIEKTQRMLSGFCPHK |
| | VSAGQFSSLHVRSSKIEVAQFVKDLLFHLRTLFRE |
| | GQFN |

### SPR Binding Results

IL-2 binds the intermediate affinity heterodimeric IL-2 receptor composed of IL2Rβ (CD122) and γ_{c} (CD132), or the high affinity heterotrimeric IL-2 receptor composed of IL2Rα (CD25), IL2Rβ and γ_{c}. MDNA209 is an IL-2 super-antagonist mutein that leads to enhanced affinity for IL2Rβ and mutations that interfere with Ye binding (Mitra et al., 2015). See also FIG. 2A.

Evaluation of binding affinity for MDNA209-Fc, MDN209FEAA-Fc, MDNA209FEAA-Fc-A11, MDNA209 (3) FEAA-Fc, MDNA209 (3) FEAA-Fc-MDNA209 (3) FEAA, MDNA209FA-Fc and MDNA209FEY-Fc variants sensorgrams are shown in FIG. 2B-D and binding summarized in Table 6. All binding K_{D}s were compared to Fc-IL-2 as a positive control. K_{D} values for MDNA209-Fc, MDNA209FA-Fc, MDNA209FEY-Fc MDN209FEAA-Fc, MDNA209FEAA-Fc-A11, MDNA209 (3) FEAA-Fc, MDNA209 (3) FEAA-Fc-MDNA209 (3) FEAA, were dramatically lower for IL2Rβ (CD122) than Fc-IL-2. Additional mutations in MDNA209FEAA-Fc, MDNA209FEY-Fc, MDNA209FA-Fc, MDNA209 (3) FEAA-Fc, MDNA209 (3) FEAA-Fc-MDNA209 (3) FEAA resulted in no binding to IL2Rα (CD25).

TABLE 6
Binding Affinity K_{D} values of IL-2 antagonists
for human CD25 and CD122

| Construct | CD25 K_{D} (nM) | CD122 K_{D} (nM) |
|---|---|---|
| Fc-IL-2 | 31.2* | 2820* |
| MDNA209-Fc | 39.1* | 3.91 |
| MDNA209FEAA-Fc | No binding | 4.94 |
| MDNA209FEAA-Fc-A11 | No binding | 4.59 |
| MDNA209(3)FEAA-Fc | No binding | 3.75 |
| MDNA209(3)FEAA-Fc- | No binding | 3.42 |
| MDNA209(3)FEAA | | |
| MDNA209FA-Fc | No binding | 5.51 |
| MDNA209FEY-Fc | No binding | 4.99 |

*K_{D} based on steady state affinity instead of typical 1:1 binding as per observed fit

### SPR Binding Conclusions

Based on SPR analyses, all the IL-2 antagonists examined demonstrated enhanced binding to CD122 compared to Fc-IL-2 (Table 6). Addition of the FEAA, FA or FEY mutations resulted in a lack of binding to CD25. In a bispecific format, MDNA209FEAA-Fc-A11 demonstrated comparable binding affinity to the MDNA209FEAA-Fc.

### Signaling Assays

### HEK-Blue™ IL-2 Reporter Antagonist Assay Methodology

HEK-Blue™ IL-2 reporter cells express the high affinity IL-2 receptor and are designed to monitor the activation of the JAK-STAT pathway. HEK-Blue™ IL-2 reporter cells were generated through stable transfection of HEK293 cells with human IL-2Rα, IL-2Rβ, IL-2Rγ, JAK3 and STAT5 genes and a STAT5-inducible SEAP (secreted embryonic alkaline phosphatase) reporter gene. IL-2 stimulation triggers the activation of STAT5 and subsequent expression and secretion of SEAP, which is quantified using QUANTI-Blue™.

HEK-Blue™ IL-2 reporter cells (InvivoGen, 50,000 cells/well) were run in two formats: 1) fixed agonist format using increasing concentrations of constructs and the EC₈₀ concentration of rhIL-2 (experimentally determined to be 0.1 nM) or 2) and antagonist format with 30 nM of test compound exposed to a range of IL-2-Fc (1 nM to 0.001 μM) (Table 7). Cells were incubated for 24 hours and after incubation, cell supernatant (20 μL) was removed to a new plate and 180 μL of QUANTI-Blue solution was added and incubated for 2 hours at 37° C. Plates were scanned on a conventional plate reader for absorbance at 650 nm.

TABLE 7
Constructs and reagents tested in HEK Blue ™ IL-2
Reporter Assay

| Construct | Source | Catalog | Concentrations |
|---|---|---|---|
| Recombinant human | Sigma | H7041-10UG | Between 1.67 nM to |
| IL-2 | | | 0.41 pM |
| IL-2-Fc | | 11977-828988 | 0.1 nM to 0.001 pM |
| MDNA209-Fc (2:1) | Abzena | Construct #8 | Between 0.006 to |
| | | | 10 nM |
| MDNA209FEAA- | Abzena | Construct #9 | Between 0.006 to |
| Fc (2:1) | | | 10 nM |
| MDNA209FEAA- | LP | TP36871F | 107 nM to 0.065 pMol |
| Fc (2:1) | | | by 4 fold dilutions |
| | | | (Agonist format) |
| MDNA209(3)FEAA- | LP | TP36872F | 107 nM to 0.065 pMol |
| Fc (2:1) | | | by 4 fold dilutions |
| | | | (Agonist format) |
| MDNA209-Fc (2:1) | Pr00532 | Pr00532 | 30 nM in the presence |
| | | | of IL-2-Fc |

### HEK-Blue IL-2 Reporter Results

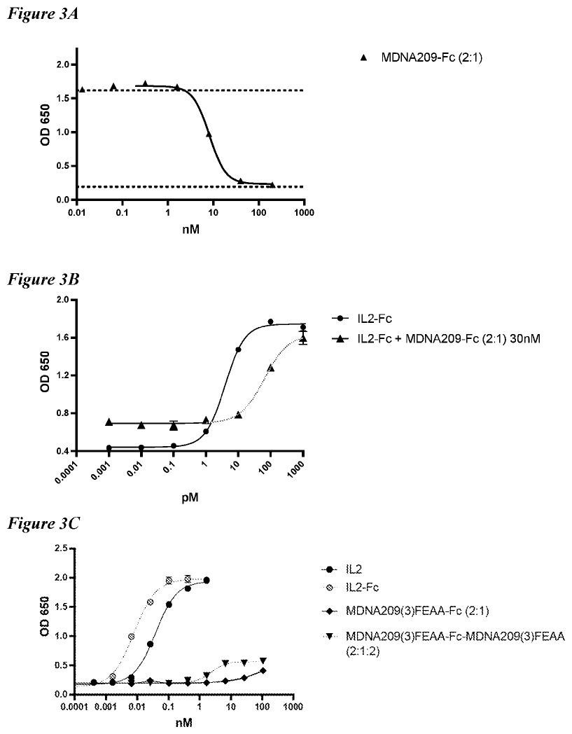

Initial experiments were performed to establish the dose of IL-2 for the antagonist assay (EC₅₀=0.025 nM). Using this assay, antagonists were screened in the HEK-Blue™ IL-2 reporter cell line and MDNA209-Fc demonstrated a clear antagonistic activity with an EC₅₀ value of 8.396 nM with a fixed agonist concentration of IL-2 (FIG. 3A and Table 7). MDNA209-Fc was also tested in an antagonist format with increasing amounts of IL-2 were added and a fixed concentration of compound. MDNA209-Fc clearly demonstrated antagonist activity as noted by a shift to the right with an IC₅₀ of 59.31 (FIG. 3B).

In a previous publication, a range of IL-2 antagonists with various level of antagonist efficacy were designed, including weak partial agonists (Mitra et al., 2015). MDNA209 (3) molecules possess three mutations (RET) that were reported to strongly but incompletely inhibit binding to γ_{c}, unlike MDNA209 (RETR) which completely abrogate γ_{c} binding. Therefore, MDNA209 (3) compounds were tested for weak partial agonist activity.

Both MDNA209 (3) FEAA-Fc and MDNA209 (3) FEAA-Fc-MDNA209 (3) FEAA demonstrated weak partial agonist activity (~60 fold less potent than free IL-2) in this assay (FIG. 3C and Table 8). MDNA209 (3)-Fc-MDNA209 (3) FEAA contains 4 MDNA209 (3) moieties and demonstrated enhanced agonist activity compared to MDNA209 (3) FEAA-Fc (FIG. 3C and Table 8).

TABLE 8
Agonist EC₅₀ Values and Antagonist IC₅₀ values in
the HEK Blue ™ IL-2 Reporter Assay

| Construct | Format | EC₅₀ (Ave ± SD) | | Fold |
|---|---|---|---|---|
| hIL-2* | Fixed Agonist | 0.04 nM | | 1.0 |
| MDNA209-Fc* | Fixed Agonist | 8.396 ± 0.294 pM | | 209.9X inhibition |
| hIL-2** | Fixed Agonist | 38.9 | pM | 1.0 |
| IL-2-Fc** | Fixed Agonist | 7.9 | pM | ~4.9X higher EC₅₀ |
| MDNA209FEAA-Fc** | Fixed Agonist | >107 | nM | ~2800X higher EC₅₀ |
| MDNA209(3)FEAA-Fc- | Fixed Agonist | 2.3 | nM | ~60X higher EC₅₀ |

| MDNA209(3)FEAA** | | | |
|---|---|---|---|
| Construct | Format | Average IC₅₀ | |
| IL2-Fc 11977-828988 | Antagonist | 3.92 (IL-2-Fc) | 1.0 |
| MDNA209-Fc (2:1) | Antagonist | 59.31 (IL-2-Fc) | 15.12X higher IC₅₀ |
| (Pr00532) | | | |

*results from experiment shown in FIG. 3A
**results from experiment shown in FIG. 3C
^{#} results from experiment shown in FIG. 3B

### HEK Blue IL-2 Reporter Conclusion

MDNA209-Fc demonstrated antagonist activity against IL-2. MDNA209 (3) FEAA-Fc and MDNA209 (3) FEAA-Fc-MDNA209 (3) FEAA demonstrated weak agonist activity.

### CTLL-2 Cell Proliferation Assay Methodology

CTLL-2 cells are a cytotoxic T cell line dependent on IL-2 for survival and proliferation. This assay provided a functional readout (proliferation) with a longer duration assay than the HEK-Blue signaling assay. The CTLL2 proliferation assay was run in duplicate on samples shown in Table 9. CTLL2 cells were plated at 30,000 cells/well in media lacking the T-STIM proliferation supplement. Cells were treated with increasing concentrations of the test or control sample for 48 hours. Cell Titer Blue viability reagent (Promega G8080) was added to each well and the plates were scanned at 560Ex/590Em after development of the fluorescent viability signal (~6 hours).

TABLE 9
Compounds Tested in the CTLL-2 Assay

| Test Article | Lot | Testing Range |
|---|---|---|
| IL-2-Fc | 11977-828988 | 3 nM to 0.011 pM by ~4 fold dilutions |
| MDNA209-Fc | Construct #8 | IL-2-Fc titration in the presence of |
| | | 30 nM |

### CTLL-2 Proliferation Results

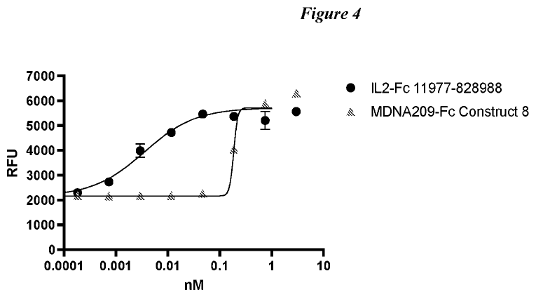

RFU versus IL-2-Fc concentration data was fit to a 4 parameter logistic curve and EC₅₀ values were calculated and the average of 2 or 4 plates (Table 10). Fold antagonism was calculated as the ratio of the IL-2-Fc EC₅₀ in the presence of test sample to the EC₅₀ of IL-2-Fc alone (FIG. 4 and Table 11). MDNA209-Fc antagonized IL-2-Fc with an EC₅₀~70 fold higher.

TABLE 10
CTLL2 EC₅₀ values (in pM).

Plate

| Compounds Tested | 1 | 2 | 3 | 4 | Avg |
|---|---|---|---|---|---|
| IL-2-Fc | 3.2 | 2.6 | 3.6 | 4.7 | 4.4 |
| MDNA209-Fc | 193.9 | 221.4 | | | 207.7 |

TABLE 11
Fold antagonism relative to IL-2-Fc.

| Compounds Tested | 1* | 2 | 3 | 4 | Avg |
|---|---|---|---|---|---|
| IL-2-Fc | 1.00 | 1.00 | 1.00 | 1.00 | 1.00 |
| MDNA209-Fc | 60.35 | 85.52 | | | 72.935 |

*The fold antagonism calculated as the EC₅₀ of the test sample divided by the EC₅₀ of IL2-Fc.

### CTLL2 Assay Conclusion

MDNA209-Fc was a potent inhibitor of IL-2 induced CTLL2 proliferation.

pSTAT5 Signaling Assay using Human Peripheral Blood Mononuclear Cells (PBMC) Methodology.

To examine the activity and potential cell population-selectivity of the various IL-2 super antagonist variants, inhibition of STAT5 phosphorylation was examined in immune cell populations (T_{reg}, CD4+ T-cells, CD8+ T-cells (resting and activated), and NK cells) present in human peripheral blood mononuclear cells (PBMCs). PBMCs purified from whole blood were rested overnight. Cells were counted and plated (60 min) in serum-free media. Media alone or containing compounds (10-point 5-fold dilutions from 25 nM) were added to the cells and incubated at 37° C. for 15 minutes. rhIL-2 (10-point 5-fold dilutions from 25 nM) was used as a positive control for pSTAT5. Following activation with IL-2 or other compounds (listed in Table 13), cells were fixed in PFA at room temperature for 15 mins and permeabilized (BD Transcription Factor Phospho Buffer Set) and stored in methanol (−20° C.). Following methanol storage, samples were stained with antibodies to detect pSTAT5, CD4, CD8, CD25, CD56 and FOXP3 and analyzed by flow cytometry. Compounds tested in the pSTAT5 Assay are shown in Table 12.

TABLE 12
Constructs tested in the pSTAT5 Assay

| | | | Catalog/ |
|---|---|---|---|
| Constructs | Description | Source | Lot |
| rhIL-2 | IL-2 control | R & D | 202-IL-010 |
| | | Systems | |
| Anti-CD122 | Neutralizing antibody | R & D | MAB224 |
| | against CD122 | Systems | |
| Anti-CD25 | Neutralizing antibody | R & D | MAB9927 |
| (Daclizumab | against CD25 | Systems | |
| biosimilar) | | | |
| MDNA209 (2:1) Fc | IL-2 antagonist | Abzena | Construct #8 |
| MDNA209FEAA-Fc | IL-2 antagonist with | Abzena | Construct #9 |
| (2:1) | additional FEAA | | |
| | mutation that inhibit | | |
| | binding to CD25 | | |
| MDNA209(3)FEAA- | Less potent IL-2 | Lake | TP36871F |
| Fc (2:1) | antagonist with | Pharma | |
| | additional FEAA | | |
| | mutation | | |
| MDNA209FEAA-Fc- | IL-2 antagonist with | Abzena | Construct |
| A11 (2:1:2) | FEAA mutation + | | #10 |
| | IL-13 agonist | | |

pSTAT5 Signaling Assay Results

Cells were pre-treated with increasing concentrations of the antagonist or neutralizing antibody control and subsequently exposed to either 67 μM or 670 μM rhIL-2 (equivalent to ~1 ng/mL and 10 ng/mL as used in the publication by Mitra et al., 2015).

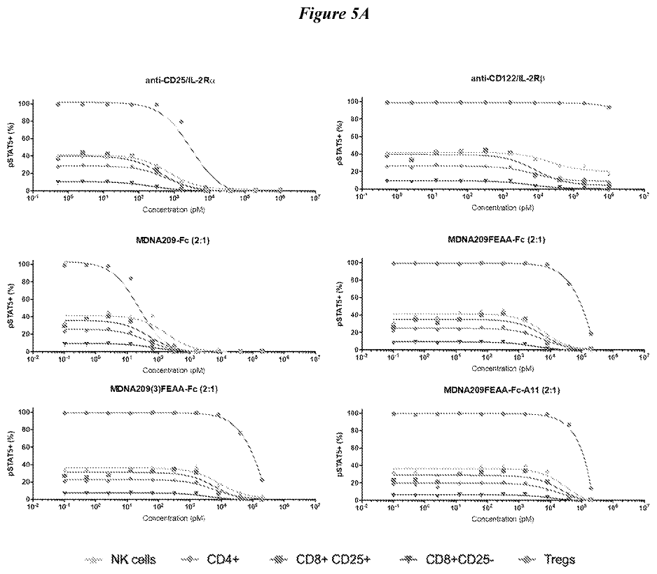

Representative dose response curves are shown in FIG. 5 and IC₅₀ summary in Tables 14 and 15. Analyses of pSTAT5 signaling in human PBMCs revealed that MDNA209-Fc demonstrated higher potency across the different cell types compared to all other antagonists and antibody controls. MDNA209-Fc appeared to also proportionally inhibit T_{reg} better than inhibition of other immune cells, including CD8+CD25+ T-cells that also expressed the high-affinity receptors. In comparison of these two cell types, anti-CD25 (Daclizumab biosimilar) appeared to have higher inhibitory activity against CD8+CD25+ T-cells than T_{reg}.

As T_{reg} cells limit T cell activity in autoimmune diseases, molecules preferentially targeting effector T cells while leaving the T_{reg} population untouched may be advantageous. Addition of the FEAA mutation which lacks binding to the high affinity CD25 receptor appeared to better separate the inhibitory effects on T_{reg} VS. other immune cells. Addition of the FEAA mutation also resulted in a significantly reduced potency towards all of the immune cell types examined, and were greater than anti-CD122. This was observed for both MDNA209FEAA-Fc and MDNA209 (3) FEAA-Fc compounds that showed similar activity levels.

Further analyses of the bispecific molecule MDNA209FEAA-Fc-A11 was done to confirm that the activity of the MDNA209FEAA moiety is not impacted by the addition of the A11 moiety. All has been shown to be inactive in this assay (data not shown), addition of a second moiety to MDNA209FEAA reduced its pSTAT5 inhibitory signaling activity.

TABLE 13
Summary IC₅₀ values and ratio analyses of % pSTAT5 positive cells in Human PBMCs (67 pM of rhIL-2).

| | | | | | | | | | CD8+ | |
|---|---|---|---|---|---|---|---|---|---|---|
| | | | | | | | | T_{reg}/ | CD25+/ | |
| | | | | | | | T_{reg}/ | CD8+ | CD8+ | CD8+ |
| Constructs/ | | NK | | CD8+ | CD8+ | | NK | CD25− | CD25− | CD25+/ |
| antibody | Donor # | cells | CD4+ | CD25+ | CD25− | T_{reg} | ratio | ratio | ratio | NK ratio |
| Anti-CD25 | 1 | 694.0 | 644.9 | 457.3 | 234.4 | 3027 | 4.7 | 12.9 | 2.0 | 0.7 |
| | 2 | 1347 | 613.9 | 629.4 | 252.8 | 3257 | 2.4 | 12.9 | 2.5 | 0.5 |
| Anti-CD122 | 1 | ND | 7167 | 8219 | 5881 | ND | — | — | 1.4 | — |
| | 2 | ND | 11097 | 11879 | 4881 | ND | — | — | 2.4 | — |
| MDNA209-Fc | 1 | 184.3 | 44.32 | 51.71 | 75.06 | 23.60 | 0.12 | 0.31 | 0.7 | 0.3 |
| | 2 | 180.9 | 59.84 | 54.93 | 128.0 | 21.82 | 0.12 | 0.17 | 0.4 | 0.3 |
| MDNA209FEAA- | 1 | 6689 | 5410 | 6541 | 2973 | ND | — | — | 2.2 | 1.0 |
| Fc | 2 | 17274 | 8509 | 10673 | 5756 | ND | — | — | 1.9 | 0.6 |
| MDNA209(3)FEAA- | 1 | 8582 | 5726 | 6534 | 3749 | ND | — | — | 1.7 | 0.8 |
| Fc | 2 | 13875 | 6338 | 8069 | 4393 | ND | — | — | 1.8 | 0.6 |
| MDNA209FEAA- | 1 | 34166 | 16132 | 22200 | 10794 | ND | — | — | 2.1 | 0.6 |
| Fc-A11 | 2 | 44038 | 13079 | 20807 | 8973 | ND | — | — | 2.3 | 0.5 |

TABLE 14
Summary IC₅₀ values and ratio analyses of % pSTAT5 positive cells in Human PBMCs (670 pM of rhIL-2).

| | | | | | | | | | CD8+ | |
|---|---|---|---|---|---|---|---|---|---|---|
| | | | | | | | | T_{reg}/ | CD25+/ | |
| | | | | | | | T_{reg}/ | CD8+ | CD8+ | CD8+ |
| Constructs/ | | NK | | CD8+ | CD8+ | | NK | CD25− | CD25− | CD25+/ |
| antibody | Donor # | cells | CD4+ | CD25+ | CD25− | T_{reg} | ratio | ratio | ratio | NK ratio |
| Anti-CD25 | 1 | ND | 1387 | 2691 | 1284 | ND | — | — | 2.1 | — |
| | 2 | ND | 2197 | 3906 | 1039 | ND | — | — | 3.8 | — |
| Anti-CD122 | 1 | ND | ND | ND | 34215 | ND | — | — | — | — |
| | 2 | ND | ND | ND | 13788 | ND | — | — | — | — |
| MDNA209-Fc | 1 | 1107 | 201.5 | 243.9 | 394.3 | 157.1 | 0.14 | 0.4 | 0.6 | 0.22 |
| | 2 | 1118 | 151.9 | 194.9 | 260.5 | 64 | 0.057 | 0.2 | 0.7 | 0.17 |
| MDNA209FEAA- | 1 | ND | ND | ND | ND | ND | — | — | — | — |
| Fc | 2 | ND | ND | ND | 11156 | ND | — | — | — | — |
| MDNA209(3)FEAA- | 1 | ND | ND | ND | ND | ND | — | — | — | — |
| Fc | 2 | ND | ND | ND | 6515 | ND | — | — | — | — |
| MDNA209FEAA- | 1 | ND | ND | ND | ND | ND | — | — | — | — |
| Fc-A11 | 2 | ND | ND | ND | 17000 | ND | — | — | — | — |

pSTAT5 Signaling Assay Conclusions

MDNA209-Fc was a potent inhibitor of pSTAT5 signaling in human PBMCs with higher proportional potency against T_{reg} than other immune cells. MDNA209FEAA-Fc demonstrated better separation of inhibitory activity on T_{reg} VS. other immune cells compared to MDNA209-Fc but also had a reduced overall potency. MDNA209 (3) FEAA-Fc and MDNA209FEAA-Fc demonstrated similar potencies in this assay. MDNA209FEAA-Fc-A11 demonstrated the least inhibitory activity observed.

### In Vivo Maximum Tolerated Dose (MTD) Methodology

The objective of this study was to determine the tolerability of MDNA209FEAA-Fc-A11 in naïve C57BL/6. An initial Maximum Tolerated Doses (MTD) study was performed to assess acute toxicity of MDNA209FEAA-Fc-A11 compound in naïve C56Bl/6 mice. A total of three mice per group were dosed with MDNA209FEAA-Fc-A11 (30 mg/kg) and A11-Fc (21 mg/kg or 11 mg/kg). Animals were dosed intraperitoneally on Day 1, 5 and 8.

### MTD Results

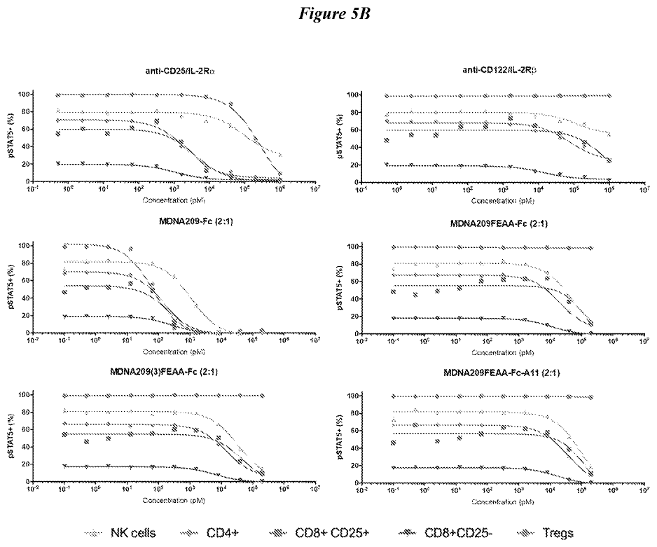

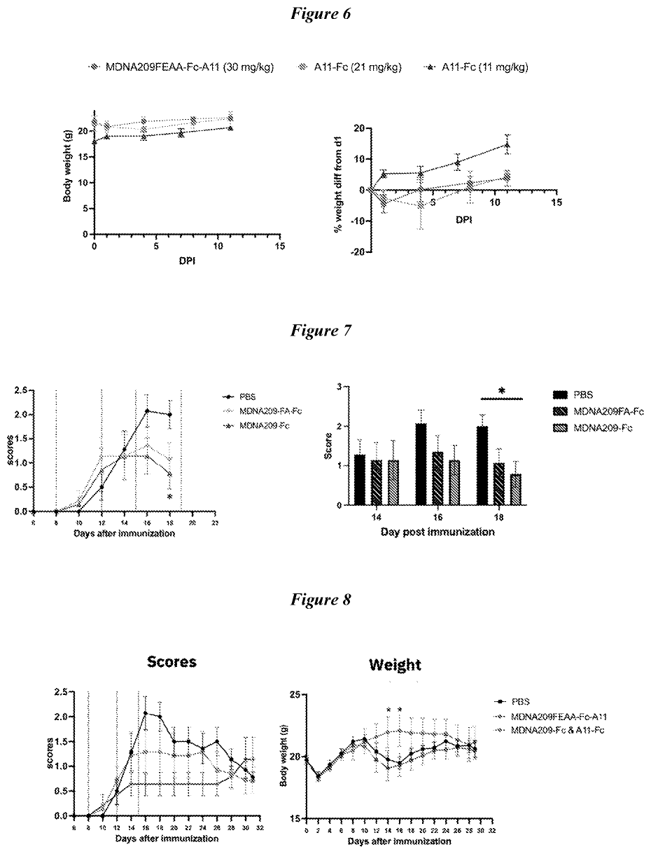

Animals receiving doses of MDNA209FEAA-Fc-A11 at 30 mg/kg) or A11-Fc (21 mg/kg, molar equivalent) did not exhibit significant weight loss or obvious signs of toxicity and tolerated the drug over a 12 day period (FIG. 6).

### MTD Conclusions

MDNA209FEAA-Fc-A11 compound was well tolerated and did not exert any signs of acute toxicity.

### Experimental Autoimmune Encephalomyelitis (EAE) Model Methodology

Experimental Autoimmune Encephalomyelitis (EAE) is an animal model of Multiple Sclerosis. C57BL/6 mice were grouped on day 0 and EAE was induced by immunization with MOG₃₅₋₅₅ peptide in complete Freund's adjuvant on day 2 at two different sites on the hind flank. Pertussis toxin was administered 2 and 48 hours after immunization. Mice were treated 8, 12 and 15 days after immunization (Table 16). Animal body weight and clinical EAE score were assessed daily and mice were scored on a scale from 0-5.0 (defined in Table 15).

TABLE 15
Clinical EAE scores

| Score | Clinical Observations |
|---|---|
| 0 | No obvious changes in motor function compared to |
| | non-immunized mice. When picked up by base |
| | of tail, the tail has tension and is erect. |
| | Hind legs are usually spread apart. When |
| | the mouse is walking, there is no gait or head tilting. |
| 0.5 | Tip of tail is limp |
| 1.0 | Limp Tail |
| 1.5 | Limp Tail and Hing Leg inhibition |
| 2.0 | Limp tail and weakness of hind legs. - OR -Mouse appears |
| | to be at score 0.0, but there are obvious signs of head |
| | tilting when the walk is observed. |
| | The balance is poor. |
| 2.5 | Limp tail and dragging of hind legs. Both hind legs have some |
| | movement, but both are dragging at the feet (mouse trips on hind |
| | feet) OR No movement in one leg but movement in the other leg |
| 3.0 | Limp tail and complete paralysis of hind legs (most common) OR |
| | Limp tail and almost complete paralysis of hind legs - One or both |
| | hind legs are able to paddle, but neither hind leg is able to move |
| | forward of the hind hip OR all of: severe head tilting, walking only |
| | along the edges of the cage, pushing against the cage wall. |
| | Spinning when picked up by the base of the tail |
| 3.5 | Mouse is moving around the cage, but when placed on its |
| | side, is unable to right itself. Hind legs are together on one side |
| | of body OR Limp tail and complete paralysis of hind legs. In |
| | addition to: Mouse is moving around the cage, but the hind |
| | quarters are flat like a pancake, giving the appearance of a |
| | hump in the front quarters of the mouse. |
| 4.0 | Limp tail, complete hind leg and partial front leg paralysis. Mouse |
| | is minimally moving around the cage but appears alert and |
| | feeding. Often euthanasia is recommended after the mouse |
| | scores 4.0 for 2 days. However, with daily s.c. fluids most |
| | C57BL/6 mice may recover to 3.5 or 3.0, while SJL mice may |
| | fully recover even if they reach score 4.0 at the peak of disease. |
| | When the mouse is euthanized because of severe paralysis, |
| | a score of 5.0 is entered for that mouse for the rest of the |
| | experiment. |
| 4.5 | Complete hind and partial front leg paralysis, no movement |
| | around the cage. Mouse is not alert. Mouse has minimal |
| | movement in the front legs. The mouse barely responds |
| | to contact. Euthanasia is recommended. When the mouse is |
| | euthanized because of severe paralysis, a score of 5.0 is entered |
| | for that mouse for the rest of the experiment. |
| 5.0 | Mouse is spontaneously rolling in the cage (euthanasia is |
| | recommended) OR Mouse is found dead due to paralysis OR |
| | Mouse is euthanized due to severe paralysis. |

TABLE 16
Dosing Groups for EAE Study

| | | | Dose | | Dosing |
|---|---|---|---|---|---|
| Test article | Lot | n | (mg/kg) | Route^{a} | frequency^{c} |
| PBS | — | 7 | N/A | i.p. | BIW |
| MDNA209FA-Fc | TP40451F | 7 | 15.15 | i.p. | BIW |
| (2:1) | | | | | |
| MDNA209-Fc (2:1) | Pr00532/T2114A16 | 7 | 15.15 | i.p. | BIW |
| MDNA209-Fc (2:1) | Pr00532/T2114A16 | 7 | 15.15 & 14.1 | i.p. | BIW |
| & A11-Fc (2:1) | Pr00546/T2118A19 | | | | |
| MDNA209FEAA-Fc- | Pr00533/T2114A17 | 7^{c} | 20^{c} | i.p. | BIW |
| A11 (2:1:2) | | | | | |

^{a}i.p.: Intraperitoneal injection
b: BIW: dosing 2 times a week. Dosing performed on days 8, 12, and 15 after immunization
^{c}therapeutic dosing group n = 5 and initial dose 20 mg/kg and subsequent doses 5 mg/kg

Mice treated with MDNA209-Fc were observed to have significantly lower scores than mice treated with PBS (p<0.05, t-test, n=7) on day 18 post immunization (FIG. 7). MDNA209FA-Fc also demonstrated a reduction in scores compared to the PBS control.

Mice were treated with MDNA209FEAA-Fc-A11 or the combination of MDNA209-Fc and A11-Fc. Both treatments resulted in a reduction in scores compared to the PBS controls (FIG. 8). Mice treated with the combination of A11-Fc and MDNA209-Fc exhibited significant increases in weight on study day 12 and 14 (p<0.05, t-test, n=6) compared to the PBS controls (FIG. 8).

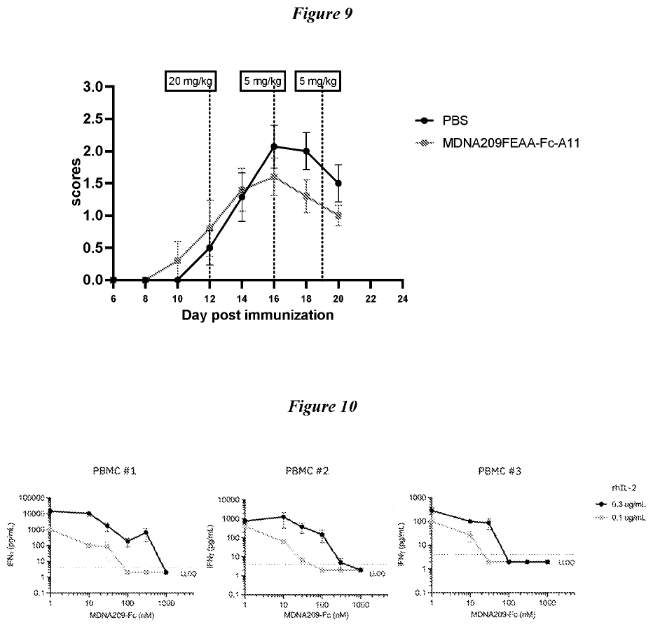

Mice receiving a therapeutic dose of MDNA209FEAA-Fc-A11 exhibited reduced scores following dosing (FIG. 9).

### EAE Conclusions

MDNA209-Fc, MDNA209FA-Fc and MDNA209FEAA-Fc-A11 reduced disease scores in the EAE model compared to PBS control animals in both prophylactic and therapeutic dosing schedules.

### Blockade of IL-2 Induced IFNγ Secretion by MDNA209-Fc

Interleukin-2 (IL-2) stimulates human peripheral mononuclear cells (PBMCs) and induces secretion of cytokines including pro-inflammatory interferon (IFN)-γ. PBMCs from 3 different healthy donors were incubated with increasing concentration of MDNA209-Fc in the presence of 0.3 or 0.1 μg/mL of human recombinant IL-2 (rhIL-2) in vitro for 48 hours. Culture supernatant was collected and analyzed for levels of IFNγ levels using an enzyme-linked immunosorbent assay (ELISA). As shown in FIG. 10, MDNA209-Fc inhibited rhIL-2 induced IFNγ secretion in a dose-dependent manner in 3 different PBMCs. Values below lower limit of quantification (LLOQ) are plotted as 0.5×LLOQ (4 μg/mL).

### Conclusions

The described MDNA209 variants were tested for: binding, signaling (HEKBlue IL-2 reporter, pSTAT5 in human PBMCs), CTLL2 proliferation, MTD in mice, and efficacy in an EAE in vivo study. All compounds showed enhanced binding to CD122 (compared to IL-2), and compounds containing FEAA, FA, or FEY mutations showed reduced binding to CD25. MDNA209-Fc demonstrated inhibitory activity in the HEKBlue IL-2 and CTLL2 assays. All compounds inhibited IL-2 induced pSTAT5 signaling in human PBMC. MDNA209FEAA-Fc-A11 was not toxic in mice (MTD assay); MDNA209-Fc reduced disease scores in EAE.

MDNA209's differentiated mechanism of action offers opportunity—MDNA209 is an antagonist of effector cells, directly targets disease-driving effector immune cells, and blocks CD4+ and CD8+ T cells and NK cells. Thus, MDNA209 has an opportunity for broad use in autoimmune indications with minimal T-reg involvement.

### Example 2: Human PBMCs Cytokine Release Assay

This study was performed to test the activity of MDNA209-Fc in blockade of IL-2 signaling in normal healthy human PBMCs using IFN-γ secretion of as a readout.

### Methodology:

PBMCs from 4 healthy human donors (Table 1) were purchased from STEMCELL Technologies and screened for their response towards IL-2 stimulation. Briefly, cells were preincubated for 15 min with 0-1000 nM MDNA209-Fc (2:1) followed by stimulation with rhIL-2 (3, 1 or 0.3 μg/ml) for 48 hrs. IFN-γ ELISA was performed on the supernatant. Since IFN-γ were above levels of quantification when PBMCs were treated with rhIL-2 at 1 and 3 μg/ml, data from 0.3 μg/ml rhIL-2 stimulation were used in the analysis and presented in this report. Data were normalized per donor by calculating the percentage inhibition of the average maximal response in the absence of MDNA209-Fc treatment.

TABLE 17
Summary of PBMC donors used in the study

| | | | | Blood | |
|---|---|---|---|---|---|
| Donor # | Gender | Age | Ethnicity | Type | smoker |
| 1 | Female | 50 | Caucasian | O+ | No |
| 2 | Female | 43 | Caucasian | B+ | No |
| 3 | Male | 20 | Caucasian | O+ | No |
| 4 | Female | 21 | Caucasian | A+ | No |

### Results and Conclusion

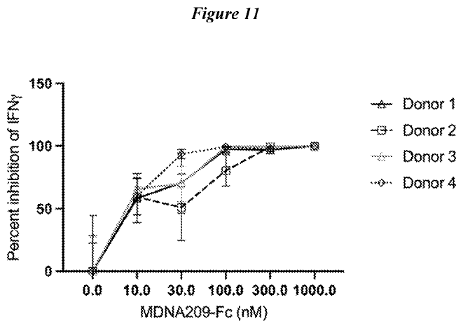

Four donors with unambiguous response to 0.3 μg/mL rhIL-2 stimulation were selected for experiments with MDNA209-Fc. In all 4 unique PMBC donor samples, MDNA209-Fc showed dose-dependent inhibition of rhIL-2 induced IFN-γ release (Table 17 and FIG. 11). Therefore, MDNA209-Fc exhibited IL-2 antagonist activity by inhibiting IL-2 induced activation and IFN-γ cytokine release in human PBMCs.

### Example 3: MDNA209 Exposure and Pharmacokinetics In Vivo

This study was performed to determine the tolerability and pharmacokinetics of MDNA209-Fc for appropriate dosing regimen in pre-clinical in vivo studies.

### Methodology:

BALB/c mice (10-11 weeks old) were randomized into 7 groups (3 animals/group) based on body weights (Table 18) and treated by intraperitoneal (IP) injection with different doses of MDNA209-Fc using different dosing schedules. Daily clinical cage-side observations and body weights twice weekly were recorded. Food and water consumption was also monitored. At the indicated non-terminal time points, approximately 100 μL of whole blood was collected and processed to plasma and stored at −80° C. until analysis. Approximately 200 μl of whole blood for CBC analysis was collected. Gross necropsy was performed upon termination. Detection of MDNA209-Fc was performed using an MDS ELISA. R&D Systems MAB202-100 was used as the capture antibody and anti-human Fc cross species absorbed (Sigma #SAB3701284) was used as the detection antibody followed by probing with HRP-conjugated anti-goat IgG (Millipore #401515).

TABLE 18
Animal study design for MDNA209-Fc dosing and blood collection

| | | | | | | CBC |
|---|---|---|---|---|---|---|
| | Test | # per | Dosage | IP treatment | PK Blood | blood |
| Group | constructs | group | (mg/kg) ^{a} | (D = day) | collection | collection |
| 1 | PBS | 3 | N/A | D 1, D 2, D 3, | 5 min after | D 5 |
| | | | | D 4 | D 1 | (terminal |
| | | | | | D 5 (terminal | bleed) |
| | | | | | bleed) | |
| e | MDNA209-Fc | 3 | 10 | D 1, D 2, D 3, | 5 min after | D 5 |
| | (2:1) | | | D 4 | D 1 | (terminal |
| | | | | | D 5 (terminal | bleed) |
| | | | | | bleed) | |
| 3 | MDNA209-Fc | 3 | 10 | D 1, D 2, D 3, | D 21 (terminal | D 21 |
| | (2:1) | | | D 4, D 8, | bleed) | (terminal |
| | | | | D 11, D 15, | | bleed) |
| | | | | D 18 | | |
| 4 | MDNA209-Fc | 3 | 10 | D 1, D 4, D 8, | D 21 (terminal | D 21 |
| | (2:1) | | | D 11, D 15, | bleed) | (terminal |
| | | | | D 18 | | bleed) |
| 5 | MDNA209-Fc | 3 | 20 | D 1, D 2, D 3, | 5 min after | D 5 |
| | (2:1) | | | D 4 | D 1 | (terminal |
| | | | | | D 5 (terminal | bleed) |
| | | | | | bleed) | |
| 6 | MDNA209-Fc | 3 | 20 | D 1, D 2, D 3, | D 21 (terminal | D 21 |
| | (2:1) | | | D 4, D 8, | bleed) | (terminal |
| | | | | D 11, D 15, | | bleed) |
| | | | | D 18 | | |
| 7 | MDNA209-Fc | 3 | 20 | D 1, D 4, D 8, | D 21 (terminal | D 21 |
| | (2:1) | | | D 11, D 15, | bleed) | (terminal |
| | | | | D 18 | | bleed) |

### Results and Conclusion

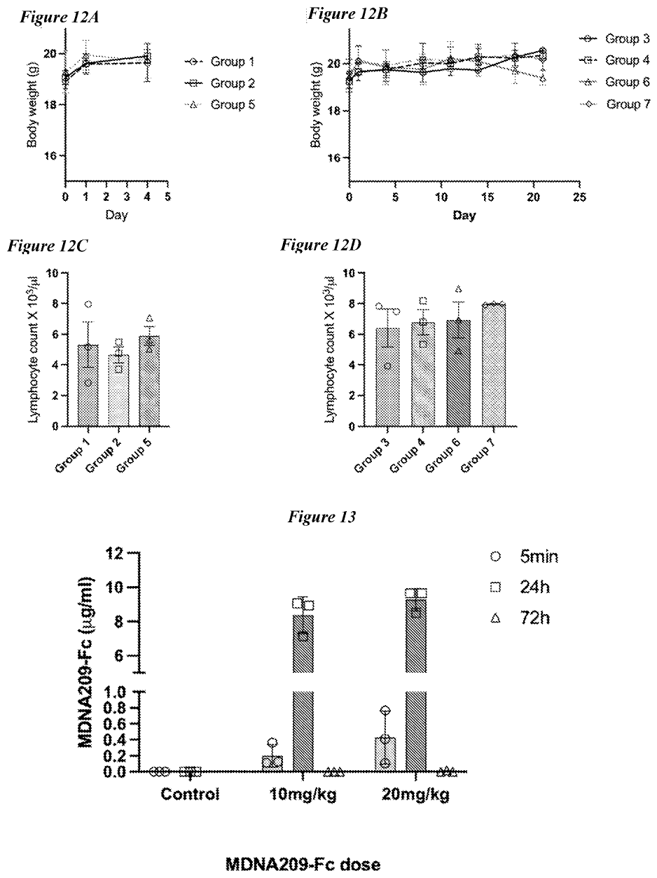

MDNA209-Fc was well tolerated when administered at repeat doses up to 20 mg/kg by IP injection in acute (4 doses in 5 days) or long-term (8 doses, 21 days) study. There were no changes in body weights and no abnormal findings at necropsy (FIG. 12). There was no effect on lymphocyte counts despite multiple administrations of MDNA209-Fc (FIG. 12), possibly due to the long-life span of lymphocytes (15-20 days), which require long-term study to detect changes. MDNA209-Fc was detectable in blood 24-hour post dose but undergoes rapid clearance in mice and was undetectable in the blood at 72-hour post dose (FIG. 13).

### Example 4: Mixed Lymphocyte Reaction (MLR) Assay

The goal of this study was to determine the efficacy of MDNA209 constructs (MDNA209-Fc, MDNA209FEAA-Fc and MDNA209-albumin) in inhibiting allogeneic Human peripheral blood mononuclear cell (PBMC) proliferation in a mixed lymphocyte reaction (MLR) assay.

### MDNA209-Albumin Protein Sequence:

(SEQ ID NO: 18)

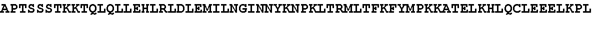

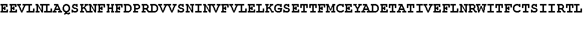

TGGGGSGGGGSGGGGSDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADKAACLLPKL
DELRDEGKASSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFVESKD
VCKNYAEAKDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCKHPEAKRM
PCAEDYLSVVLNQLCVLHEKTPVSDRVTKCCTESLVNRRPCFSALEVDETYVPKEFNAETFTFHAD
ICTLSEKERQIKKQTALVELVKHKPKATEQLKAVMDDFAAFVEKCCKADDKETCFAEEGKKLVAA
SQAALGL

Linker sequence is italic and underlined.
MDNA209 sequence is in bold. The mutations from wild type human IL-2 are highlighted with grey and were as follows: L18R, Q22E, L80F, R81D, L85V, I86V, I92F, Q126T, S130R
Albumin sequence is in normal font.

### Methodology:

Four healthy donors were sourced by Xeno Diagnostics for use in this study (Table 19). Blood samples were collected by venipuncture and PBMCs were isolated by Ficoll density gradient centrifugation and cryopreserved. Prior to use, the cryopreserved PBMCs were thawed and rested overnight. PBMCs from two unrelated donors (200K cells/donor) were co-cultured with different dilutions (0.0016 nM-100 nM; 5-fold dilutions) of MDNA209-Fc, MDNA209-albumin and MDNA209FEAAC-Fc. The mitogenic stimulator phytohemagglutinin (PHA) and the proliferation inhibitor dexamethasone, were used as positive and negative controls respectively. Samples were performed in triplicate and cultured for 4 days. On Day 3, proliferation was microscopically observed and BrdU was added. On day 4 BrdU incorporation colorimetric ELISA was performed. Stimulation index (SI) was calculated by dividing the test absorbance by the absorbance of the baseline background controls. Half maximal inhibitory concentration (IC₅₀) was calculated for SI with 4-parameter curve fitting.

TABLE 19
PBMC donor samples used in the study

| Donor # | Gender | Age | Race/Ethnicity |
|---|---|---|---|
| 50 | Male | 30 | African American |
| 59 | Female | 37 | Caucasian |
| 90 | Male | 31 | Caucasian |
| 80 | Male | 26 | Hispanic |

### Results and Conclusion

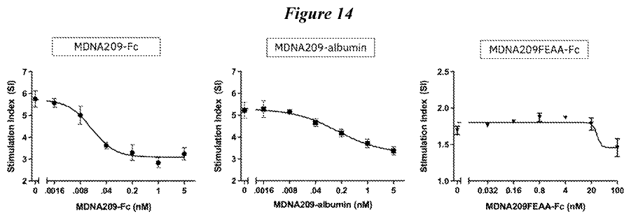

MDNA209-Fc inhibited PBMC proliferation in all 5 donor pairs (IC₅₀=0.0294±0.01 nM). While MDNA209FEAA-Fc was inhibitory only in one pair (IC₅₀=28.54 nM), MDNA209-albumin was able to inhibit PBMC proliferation (IC₅₀=0.18±0.02 nM) in two test pairs, out of three, within the tested dose range. Overall, MDNA209-Fc was 15-fold more potent than MDNA209-albumin at blockade of proliferation in an MLR setting. MDNA209FEAA-Fc had limited to no effect on PBMC proliferation. See FIG. 14 and Table 20.

TABLE 20
IC50 values and fold potency for MLR assay for tested constructs

| | IC₅₀ (nM) for donor pairs # | | | | | Average |
|---|---|---|---|---|---|---|
| Constructs Tested | 1 | 2 | 3 | 4 | 5 | IC₅₀ (nM) |
| MDNA209-Fc | 0.054 | 0.011 | 0.054 | 0.012 | 0.016 | 0.0294 |
| MDNA209FEAA- | >100* | 28.54 | >100* | not tested | not tested | 28.54 |
| Fc | | | | | | |
| MDNA209- | not tested | 0.202 | not tested | 0.158 | >5* | 0.179 |
| albumin | | | | | | |

fold-change^{#} in potency

| | | | | | | |
|---|---|---|---|---|---|---|
| MDNA209Fc vs | NA | 2595X | NA | NA | NA | 2595X |
| MDNA209FEAA- | | | | | | |
| Fc | | | | | | |
| MDNA209Fc vs | NA | 18X | NA | 13X | NA | 15.5X |
| MDNA209- | | | | | | |
| albumin | | | | | | |

*maximum tested concentration
^{#}fold change in IC₅₀ value was calculated with respect to same donor pair

### Example 5: Further Binding (SPR) Data

### Results and Conclusion

Based on SPR analyses, all the IL-2 antagonists (free and Fc fused) examined demonstrated enhanced binding to CD122 compared to respective IL-2 or Fc-IL-2 controls (Table 21). Addition of the FEAA resulted in a lack of binding to CD25. MDNA209 (3)-Fc retained binding to CD25 and enhanced binding to CD122. Addition of FA to MDNA209 (3) reduced its affinity to CD25.

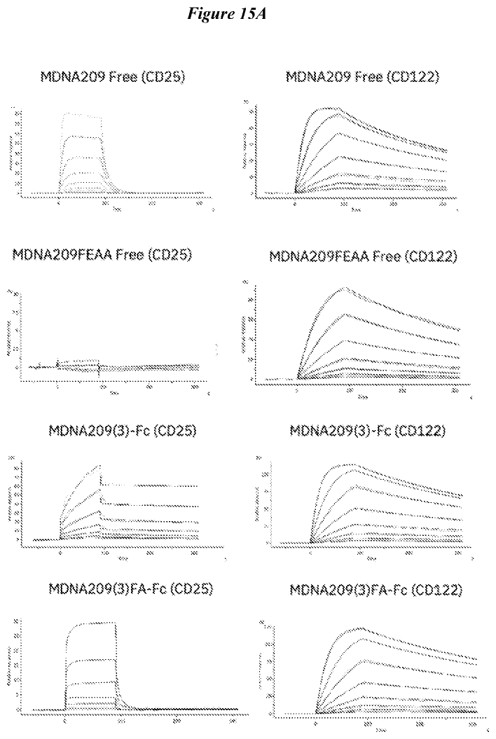

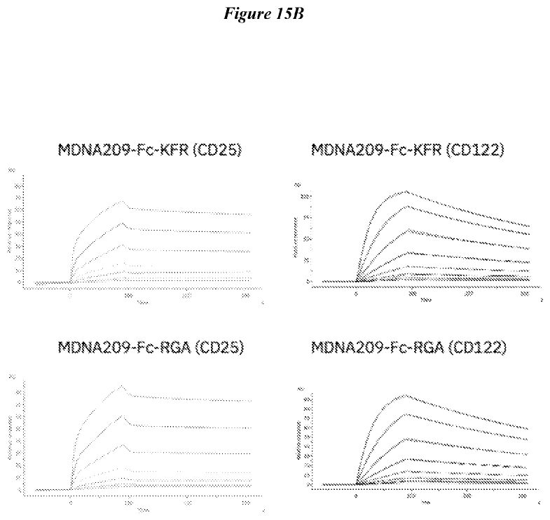

Fusion of MDNA209 to IL-4 agonists KFR or RGA moieties maintained the binding affinities to CD25 and CD122 (Table 21, FIGS. 15A-15B).

TABLE 21
Binding Affinity KD values of IL-2 antagonists for
human CD25 (IL-2Rα) and CD122 (IL-2 Rβ)

| | | | K_{D} (nM) | |
|---|---|---|---|---|
| S. no. | Construct | source | IL-2Rα | IL-2 Rβ |
| 1 | Human IL-2 | E. coli | 21.6 | 408 |
| 2 | MDNA209 (free) | CHO | 31.3 | 0.537 |
| 3 | MDNA209FEAA (free) | CHO | No binding | 1.59 |
| 4 | MDNA209(3)-Fc | CHO | 35.8 | 3.25 |
| 5 | MDNA209(3)FA-Fc | CHO | 4610* | 3.79 |
| 6 | MDNA209-Fc-KFR | CHO | 32.2* | 5.02 |
| 7 | MDNA209-Fc-RGA | CHO | 40.3* | 6.03 |

*K_{D} based on steady state affinity instead of typical 1:1 binding as per observed fit

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An IL-2 mutein comprising amino acid substitutions L18R, Q22E, and Q126T numbered in accordance with wild-type human IL-2 (hIL-2) (SEQ ID NO: 8), and further comprising a group of amino acid substitutions selected from the group consisting of F42A, Y45A, E62A, L80F, R81D, L85V, I86V, I92F, Q126T, and S130R.

2. The IL-2 mutein of claim 1, wherein the IL-2 mutein comprises the amino acid substitutions L18R, Q22E, L80F, R81D, L85V, I86V, I92F, Q126T, and S130R, optionally wherein the IL-2 mutein comprises an amino acid sequence of SEQ ID NO: 1.

3. The IL-2 mutein of claim 1, wherein the IL-2 mutein comprises the amino acid substitutions L18R, Q22E, L80F, R81D, L85V, I86V, I92F, Q126T, S130R, F42A, and E62A, optionally wherein the IL-2 mutein comprises an amino acid sequence of SEQ ID NO: 2.

4. The IL-2 mutein of claim 1, wherein the IL-2 mutein comprises the amino acid substitutions L18R, Q22E, L80F, R81D, L85V, I86V, I92F, Q126T, S130R, and F42A, optionally wherein the IL-2 mutein comprises an amino acid sequence of SEQ ID NO: 3.

5. The IL-2 mutein of claim 1, wherein the IL-2 mutein comprises the amino acid substitutions L18R, Q22E, L80F, R81D, L85V, I86V, I92F, Q126T, S130R, F42A, and Y45A, optionally wherein the IL-2 mutein comprises an amino acid sequence of SEQ ID NO: 4.

6. The IL-2 mutein of claim 1, wherein the IL-2 mutein comprises the amino acid substitutions L18R, Q22E, L80F, R81D, L85V, I86V, I92F, and Q126T, optionally wherein the IL-2 mutein comprises an amino acid sequence of SEQ ID NO: 5.

7. The IL-2 mutein of claim 1, wherein the IL-2 mutein comprises the amino acid substitutions L18R, Q22E, L80F, R81D, L85V, I86V, I92F, Q126T, F42A, and E62A, optionally wherein the IL-2 mutein comprises an amino acid sequence of SEQ ID NO: 6.

8. The IL-2 mutein of claim 1, wherein the IL-2 mutein comprises the amino acid substitutions L18R, Q22E, L80F, R81D, L85V, I86V, I92F, Q126T, and F42A, optionally wherein the IL-2 mutein comprises an amino acid sequence of SEQ ID NO: 7.

9. The IL-2 mutein of claim 1, wherein the IL-2 mutein is fused to an albumin molecule, an Fc molecule, and/or another mutein, optionally wherein the other mutein is an IL-13 mutein or an IL-4 mutein.

10. The IL-2 mutein of claim 9, wherein the IL-2 mutein is fused to an albumin molecule.

11. The IL-2 mutein of claim 10, wherein the fusion protein comprises an amino acid sequence of SEQ ID NO: 18.

12. The IL-2 mutein of claim 9, wherein the IL-2 mutein is fused to an Fc molecule.

13. The IL-2 mutein of claim 9, wherein the IL-2 mutein is fused to an Fc molecule and an IL-13 mutein, optionally wherein the IL-13 mutein comprises the amino acid substitutions L10V, V181, D87S, T88S, L101F, K104R, and K105T (A11) numbered in accordance with wild-type human IL-13 (hIL-13).

14. The IL-2 mutein of claim 9, wherein the IL-2 mutein is fused to an Fc molecule and an IL-4 mutein, optionally wherein the IL-4 mutein comprises the amino acid substitutions R121K, Y124F, and S125R (KFR) or K117R, T118V, R121Q, E122S, Y124W, S125F, S128G, and S129A (RGA) numbered in accordance with wild-type human IL-4 (hIL-4).

15. The IL-2 mutein of claim 14, wherein the fusion protein comprises an amino acid sequence of one of SEQ ID NOs: 19-22.

16. The IL-2 mutein of claim 1, wherein
i) the IL-2 mutein has increased binding to CD122, compared to wild-type IL-2;
ii) the IL-2 mutein has decreased binding to CD25, compared to wild-type IL-2;
iii) the IL-2 mutein has inhibitory activity, as determined using a HEKBlue IL-2 and/or a CTLL2 assay;
iv) the IL-2 mutein inhibits IL-2 induced pSTAT5 signaling in human PBMCs;
v) the IL-2 mutein is not toxic in mice, as determined using a maximum tolerated dose (MTD) assay; and/or
vi) the IL-2 mutein reduces disease scores, as determined using Experimental Autoimmune Encephalomyelitis (EAE) analysis.

17.-21. (canceled)

22. A nucleic acid encoding the IL-2 mutein of claim 1.

23. A vector comprising the nucleic acid of claim 22.

24. A host cell comprising the nucleic acid of claim 22 or the vector of claim 23.
